(19) United States
(12) Patent Application Publication
(10) Pub. No.: US 2011/0307079 A1
(43) Pub. Date: Dec. 15, 2011

(54) MULTISCALE INTRA-CORTICAL NEURAL INTERFACE SYSTEM

(75) Inventors: Karim Oweiss, Okemos, MI (US); Mehdi Aghogolzadeh, East Lansing, MI (US)

(73) Assignee: BOARD OF TRUSTEES OF MICHIGAN STATE UNIVERSITY, THE, EAST LANSING, MI (US)

(21) Appl. No.: 13/098,376

(22) Filed: Apr. 29, 2011

Related U.S. Application Data
(60) Provisional application No. 61/329,437, filed on Apr. 29, 2010.

(51) Int. Cl. A61B 5/0482; A61F 2/60; A61F 2/54; A61M 5/168; A61F 2/00

(52) U.S. Cl. 623/27; 600/545; 604/66; 623/66.1; 623/57

(57) ABSTRACT

Apparatus, systems, and methods may operate to collect neuro data from an organ, such as a brain. Spikes may be detected using raw neuro data collected from the organ. The spikes may be sorted. Underlying neuronal firing rates may be estimated using the sorted spikes. The neuronal firing rates may be transmitted outside the organ for real time decoding.

The present application claims the priority benefit under 35 U.S.C. 119(e) of U.S. Provisional Patent Application Ser. No. 61/329,437, filed Apr. 29, 2010, and entitled “MULTISCALE INTRA-CORTICAL NEURAL INTERFACE SYSTEM,” of which application is incorporated herein by reference in its entirety.

### STATEMENT OF GOVERNMENT SUPPORT

This invention was made with government support under 1 R01-NS-062031-01A1 awarded by the National Institute of Neurological Disorders and Stroke. The government has certain rights in the invention.

### COPYRIGHT

A portion of the disclosure of this document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software, data, and/or screenshots that may be described below and in the drawings that form a part of this document: Copyright © 2011, Michigan State University. All Rights Reserved.

### BACKGROUND

Recent technological and scientific advances have generated wide interest in the possibility of creating brain-machine interfaces (BMI) as a means to aid paralyzed humans in communication and daily activities. Advances have been made in detecting neural signals and translating them into command signals that can control devices. Devices such as these are potentially valuable for restoring lost neurological functions associated with spinal cord injury, degenerative muscular diseases, stroke, or other nervous system injury. While efforts are underway to develop BMI systems that translate neural signals from the cortex to usable output data, the limitations of current neural data acquisition technologies require subjects to be tethered to large equipment thus hindering the potential clinical applications.

BMI systems may help alleviate the presently estimated nerve injury cost statistics of approximately $7 billion annually in the U.S. alone (American Paralysis Association, 1997). These costs are reflected in current 250,000 Americans (approximately 11,000 per year) having spinal cord injuries, wherein 52% of spinal cord injured individuals are considered paraplegic and 47% quadriplegic.

From the neural data acquisition standpoint, many companies sell systems that feature racks of equipment to perform the signal processing tasks designed for rehabilitation devices and/or prosthetic devices. These systems are bulky and wired, requiring the subject to be tethered to the recording device for a large number of hours leading to fatigue and exhaustion that can significantly impact the type of brain signals being recorded.

### BRIEF DESCRIPTION OF THE DRAWINGS

FIG. 1A shows a schematic for a general brain-machine interface device according to various embodiments.

FIG. 1B shows a schematic for a neural interface node (NIN) and a manager interface module (MIM) according to various embodiments.

FIG. 2 shows a schematic illustration of a BMI ‘brain pacemaker’ that monitors neural activity using a VLSI chip designed to detect seizure activity.

FIG. 3 shows an HBMI for controlling a robotic prosthetic arm using brain-derived signals.

FIG. 4 shows an organization of a brain-machine interface (BMI) according to various embodiments.

FIG. 5 shows examples of intracortical electrode arrays; (a) a commercially available Silicon 100 electrode array; each is separated by 400 μm (Blackrock Microsystems); (b) a silicon array shown against a penny (US) to illustrate size; (c) a thin film 256-shank array of 1024 multiplexed sites with mounted signal processing electronics; and (d) a silicon array shown again a finger tip.

FIG. 6 shows a schematic diagram of a data flow in a neuromotor prosthetic application, including a data flow according to various embodiments.

FIG. 7 shows a) a representation of sample events from three units, “A,” “B,” and “C” in the noiseless (middle) and noisy (right) neural trace for five wavelet decomposition levels indicated by the binary tree (left) according to various embodiments. First level high-pass coefficients (node 2) are omitted as they contain no information in the spectral band of spike waveforms. Sensing thresholds are set to allow only one feature/event to survive in a given node. In this case, it is a local average of 32/2^{j} coefficients. For example, nodes 4 and 6 can either be used to mark events from unit “B,” while node 9 can be used to mark events from unit “A.” When noise is present (right), the sensing threshold also serves as a denoising one and (b) exemplary data of 1-D and 2-D joint distributions of wavelet features for nodes 9 and 10 for the three units over many spike occurrences from each unit showing three distinct clusters according to various embodiments. These projections can be used when spikes from different units result in identical sparse representations in a particular node (e.g., node 10). This can be used to resolve the ambiguity provided that these units were not already discriminated in earlier nodes.

FIG. 8 shows five units obtained from spontaneous recordings in an anesthetized rat preparation according to various embodiments. Units were chosen to possess significant correlation among their spike waveforms as seen in the PCA feature space in (c). (a) Events from each recorded unit, aligned and superimposed on top of each other for comparison. (b) Corresponding spike templates obtained by averaging all events from each unit on the left panel. (c) PCA 2-D feature space. Dimensions represent the projection of spike events onto the two largest principal components. (d) Clustering result of manual, extensive, offline sorting using hierarchical clustering using all features in the data. (e) Clustering result using the two largest principal components and EM cluster-cutting based on Gaussian mixture models. This is an example of a suboptimal sorting method with relatively unlimited computational power.

FIG. 9A shows a unit isolation quality of the data in FIG. 8 according to an example embodiment. Each cell in the left side shows the separation (displayed as a 2-D feature space for illustration only) obtained using the compressed sensing method. The highest magnitude coefficients that survive the sensing threshold in a given node are considered irregular samples of the underlying unit's firing rate and are marked with the “Gold” symbols in the left panel. The feature space of the sorted spikes using the manual, extensive, offline spike sorting is re-displayed in the right side (illustrated with the same color code as FIG. 8) for comparison. If a gold cluster from the left panel matches a single colored cluster from the right panel in any given row, this implies that the corresponding unit is well isolated in this node using the single feature/event magnitude alone. The unit is then removed from the data before subsequent DWT calculation is performed in the next time scale. Using this approach, three out of five units (pink, red, and green) in the original data were isolated during the first iteration in nodes 4, 6, and 9, respectively, leaving out two units to be isolated with one additional iteration on node 9's remaining coefficients. In the first iteration, node 2 shows weak separation (SR=0.45) between units. Unit 4 has larger separability in node 4 (SR=1.07). Units 1 and 2 are separated in nodes 6 and 9 (SR=1.15 and 1.51, respectively). Units 3 and 5 are separated in node 9 afterwards (SR=1.14). (b) Quantitative analysis of spike class separability versus number of coefficients retained per event (40 coefficients retained implies 0% compression of the spike waveforms, while 1 coefficient retained implies 100% compression) (i.e., thresholding) for 24 units recorded in the primary motor cortex of anesthetized rat. A 2.5 dB (>75%) improvement can be observed when the two most significant coefficients are averaged compared to time domain separability.

FIG. 9B shows a compressive sorting module output during the “sensing mode” operation according to various embodiments: (a) Top row: actual recording (black), and the reconstruction (red). Following rows: the wavelet-tree decomposition of nodes d2, d3, d4 and a4, respectively. Surviving coefficients are represented by red dots; and (b) The two dimensional feature space of the spike waveforms from three neurons (red, green and blue circles). Events that pass the neuron-specific threshold are represented as filled circles.

FIG. 10 shows various embodiments including (a) a schematic of encoding 2-D, nongoal-directed arm movement: the sample network of neurons is randomly connected with positive (excitatory), and negative (inhibitory) connections. Right panel demonstrates a symbolic movement trajectory to indicate the movement parameter encoded in the neural population model. Sample firing rates and corresponding spike trains are shown to illustrate the distinct firing patterns that would be obtained with broad and sharp tuning characteristics. (b) Sample tuning characteristics (over a partial range) of a subset of the 50 neurons modeled with randomly chosen directions and widths. (c) Sample 3-s raster plot of spike trains obtained from the population model.

FIG. 11 shows various embodiments including (a) Top-left: 400 ms segment of angular direction from a movement trajectory superimposed on tuning “bands” of five representative units. Top right, middle, and bottom panels: Firing rates obtained from the point process model for five units and their extended DWT (EDWT), Gaussian, and rectangular kernel estimators. As expected, the rectangular kernel estimator is the noisiest, while the Gaussian and EDWT estimators are closest to the true rates. (b) Mean square error between the actual (solid black line) and the estimated firing rate for each neuron with the three methods. Each pair of dotted and dashed lines is the MSE for rectangular and Gaussian kernel methods, respectively, for the five units in FIG. 11A. These remain flat as they do not depend on the DWT kernel window length. For the sharply tuned neurons, on average, ten levels of decomposition result in a minimum MSE that is lower than the MSE for rectangular and Gaussian kernel methods. For broadly tuned neurons, 12 levels of decomposition result in optimal performance. (c) Tuning width versus optimal kernel size. As the tuning broadens, larger kernel windows (i.e., coarser time scales) are needed to obtain optimal rate estimators.

FIG. 12 shows average mutual information (in bits) between movement direction, θ, and rate estimators averaged across the two subgroups of neurons in the entire population as a function of decomposition level (i.e., kernel size) according to various embodiments. Solid lines indicate the performance of the EDWT method (dark for the broad tuning group and gray for the sharp tuning group). The two dashed lines represent the Gaussian kernel method (broad tuning and sharp tuning groups), while the two dotted lines represent the rectangular kernel method in a similar way. As expected, sharply tuned neurons require smaller kernel size to estimate their firing rates. Overall, the EDWT method achieves higher mutual information than either the fixed width Gaussian or rectangular kernels for broadly tuned neurons, while slightly less for sharply tuned neurons owing to the relatively more limited response time these neurons have, limiting the amount of data.

FIG. 13 shows decoding performance of a sample 2-D movement trajectory according to an embodiment. The black line is the average over 20 trials, while the gray shade around the trajectory represents the estimate variance. Top left: one unit is observed on any given electrode (i.e., neural yield=1) and therefore no spike sorting is required. The variance observed is due to the network interaction. Top right: every electrode records two units on average (neural yield=2) and no spike sorting is performed. Bottom left: PCA/EM/Gaussian kernel spike sorting and rate estimation is implemented. Bottom right: Compressed sensing decoding result.

FIG. 14 shows computational complexity of PCA/EM/Gaussian kernel and the compressed sensing method according to various embodiments: (a) Computations per event versus number of events and number of samples per event in the training mode. (b) Computations per event versus number of samples per event and kernel size in the runtime mode. At a sampling rate of 40 KHz and ˜1.2-1.5 ms event duration (48-60 samples), the compressed sensing method requires less computations than the PCA/EM/Gaussian kernel method. The number of units is assumed fixed in the training mode for both methods (P=50).

FIGS. 15A and 15B each show a schematic diagram of an implantable system comprising a compressive spike sorting module according to various embodiments: FIG. 15A presents a system diagram for a NIN and its operational modes. FIG. 15B presents a system diagram for the MIM.

FIG. 16 shows channel activity and data exchange at different states for 4-level DWT according to various embodiments.

FIG. 17A presents data structure for a uplink data packet and downlink command packet according to various embodiments.

FIG. 17B shows a spike sorting output of the thresholding block for a sample neural trace with three distinct spike shapes presumably belonging to three distinct cells using DWT coefficients according to various embodiments. Events surpassing the node-specific thresholds are transmitted to an external observer in a 26-bit packet format. At the destination, spike event ‘y’ is detected at node 8, followed by ‘x’ at node 6, and ‘z’ at node 4.

FIG. 17C shows ROC curves for different bit precisions according to various embodiments. The performance improvement for =>10 is negligible.

FIG. 18 shows an implantable wireless transmission module according to various embodiments; the convolutional encoder, packetizer and the memory block are parts of the digital core.

FIG. 19 illustrates a birth-death process, characterized by the mean arrival and mean service rates according to various embodiments; state Pₖ can only transit to either Pₖ₋₁ or Pₖ₊₁.

FIG. 20 shows an overhead introduced by encoding and packetizing the input data stream according to various embodiments.

FIG. 21 illustrates a finite-state Markov channel with two levels of mobility, the rest and active states; each state has a particular binary error rate, ρ, according to various embodiments.

FIG. 22 presents simulation of a noisy wireless channel with time-varying binary error rate according to various embodiments: The top raster plot shows in-vivo recordings from the barrel cortex of a rat. The bottom raster plot shows the reconstruction of the in-vivo recordings, after correcting the contaminating errors, introduced through the wireless channel.

FIG. 23 presents a 7th-order convolutional encoder according to various embodiments: x[n] is the input data stream, and y₁[n] and y_{z}[n] are the encoded output streams associated with different generator functions. The data rate in this case is 0.5.

FIG. 24 shows a relation between a number of uncorrectable errors and a binary error rate for different packet lengths according to example embodiments. The middle line is the average number of uncorrected errors for each packet length, and the shaded region around it is the standard deviation. The dotted line indicates that up to one uncorrected error is acceptable. This can be varied by the user depending on the application at hand.

FIG. 25 shows a relation between a maximum number of correctable errors and a packet length according to various embodiments:

FIG. 26 shows average memory length versus packet length for different BER according to various embodiments. The minimum for each BER indicates the optimal average memory length for the corresponding packet length.

FIG. 27 shows a flow diagram of various methods according to various embodiments.

FIG. 28 shows a block diagram of a system according to example embodiments.

FIG. 29 shows an article of manufacture, including a storage device, which may store instructions to perform methods according to various embodiments.

### DETAILED DESCRIPTION

What is needed in the art is a simple, low power device capable of real time neural data reduction and wireless transmission that control medical devices (i.e., for example, pharmaceutical mini-pumps or prosthetic devices) by brain motor intention signals.

In one example embodiment, a method for transmitting neural signals from brain cells using ultra-high communication bandwidths is disclosed. Furthermore, in one example embodiment, methods of extracting information reliably from neural signals to characterize brain function are disclosed. For example, such neural information may be derived from healthy normal neurons and/or from neurons exhibiting neurological diseases and/or disorders including, but not limited to, Parkinson's disease and/or epilepsy. These method can be integrated into brain-machine interfaces for treating severe paralysis (i.e., for example, that caused by spinal cord injury), artificial prosthetic control, and/or detecting/preventing sudden onset neuronal afflictions (i.e., for example, seizures).

In one example embodiment, a fully wireless brain-machine instrument for continuously acquiring and processing neural data signals is provided. In one embodiment, the instrument provides continuous monitoring of neural signals at exceedingly high resolution over a single cell or large distributed cell population. In one embodiment, instrument comprises at least two modules, wherein the first module comprises a subcutaneously implanted chip capable of front end signal processing, information extraction and data compression, and a second module capable of transmitting the neural information to a central base station for further analysis.

In one example embodiment, this instrument solves known problems associated with ultra-high communication bandwidth requirements for the transmission of neural signals from brain cells to an external recording device. It is further believed that wireless transmission of the neural data from the second module to the base station allow subjects to be unrestrained, untethered, and freely interacting with the surrounding environment. In one embodiment, the system comprises a subcutaneously implanted chip (i.e., for example, a NIN module) featuring front end signal processing, information extraction and data compression, and a transmitter (i.e., for example, a MIM module) fixated extra-cranially to relay the information from the NIN module to a central base station for further analysis.

### Definitions

The term “microchip” as used herein, refers to a solid substrate comprising a semiconducting material, generally in the shape of a square a few millimeters long, cut from a larger wafer of the material, on which a transistor or an entire integrated circuit is formed.

The term “biocompatible”, as used herein, refers to a material which does not elicit a substantial detrimental response in the host. When a foreign object is introduced into a living body, the object may induce an immune reaction, such as an inflammatory response that will have negative effects on the host.

The term “compressive spike sorting module”, as used herein, refers to an algorithm, or series of algorithms, that processes neural spike train data in a real time manner and may be transmitted by wireless devices.

The term “transmitter”, as used herein, refers to a device capable of receiving and sending electronic information. Such transmitters may be connected to other electronic devices using wires and/or cables (i.e., hard wired) or capable of ‘wireless’ transmission using, for example, electromagnetic waves.

The term “electronically connected”, as used herein, refers to a link between a sending and receiving device such that information is reliably transmitter. For example, an electronic connection may comprise ‘high density contacts’, exemplified by soldered pathways or a network of wires (i.e., for example, microwires) from one device to another device. Alternatively, an electronic connection may be wireless.

The term “microelectrode” or “microelectrode array” as used herein, refers to a sensor capable of detecting and transmitting electrical fields in and around biological cells (i.e., for example, a neuron) to a recording device (i.e., for example, a microchip). As exemplified herein, microelectrodes may be used to detect and transmit neural spike trains that comprise information regarding neuron action potentials.

The term “discrete wavelet transform block” as used herein, refers to an algorithm capable of discrete wavelet transform (DWT) calculations. DWT is utilized to decompose a spike waveform during a sparse representation analysis that can obtain single features within a spike waveform. Such features include but are not limited to, spike times and/or spike shape.

The term “thresholding block” as used herein, refers to an algorithm capable of processing DWT data such that specific identifying indices are extracted that code neural data. Such indices may include, but are not limited to, a channel index, a node index, or a time index.

The term “packet formatter block” as used herein refers to an algorithm that codes neural data using various indices identified by the thresholding block analysis.

The term “a base station”, as used herein, refers to a device that is physically separated from a patient who is capable of receiving processed neural data from a transmitter. The base station may be capable of receiving hard wired data, or wireless data. For example, a base station may be a desktop microprocessor or other type of computer.

The term “patient”, as used herein, refers to a human or animal and need not be hospitalized. For example, out-patients and persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “neural data signals” as used herein, refers to an electromagnetic signals generated by cells of a biological nervous system. Typically, such signals comprise neuronal spike train signals that are representative of action potentials.

The term “recorded” or “recording” as used herein, refers to a process where electronic information is fixed on a media (i.e., for example, a microchip) such that the information may be accessed and processed with other recorded data.

The term “extracting” as used herein, refers to an algorithm capable of mathematically identifying unique indices within neural data signals. For example, the unique indices may represent a command signal that initiates muscular control for movement of an appendage and/or prosthetic medical device. Alternatively, the command signal may trigger deep brain stimulation by a stimulator medical device.

The term “formatting” as used herein refers to a method by which specific coding information is selected and packaged that provide a unique identification of neural information (i.e., for example, at least one index value) that is at least 90% reduced in bandwidth than the raw data stream. Such index values are combined in “packets” wherein each packet represents a specific portion of the raw data stream (i.e., for example, a command signal).

The term “real time” as used herein, refers to the near instantaneous transformation of information from one state to another. Such transformations may include, but are not limited to, collecting, processing, extracting, formatting, and/or transmitting (i.e., for example, wirelessly) of neural data signals collected from a living organism such that a medical device may be moved and/or activated within milliseconds of neural data signal collection.

The term “neural spike train” as used herein, refers to a pattern of neural data signals showing periodic sharp increases and/or decreases in electrical voltages. Such changes in voltages may be decoded by DWT to extract and identify specific neural information reflective of mental intentions (i.e., for example, movement intentions).

The term, “action potential” as used herein, refers to a change in electrical potential that occurs between the inside and outside of a nerve or muscle fiber when it is stimulated, serving to transmit nerve signals.

The term “medical device”, as used herein, refers broadly to an apparatus used in relation to a medical procedure and/or medical treatment. Specifically, the term “medical device” refers to an apparatus that contacts a patient during a medical procedure or therapy as well as an apparatus that administers a compound or drug to a patient during a medical procedure or therapy. “Direct medical implants” include, but are not limited to, drug delivery mini-pumps, urinary and intravascular catheters, dialysis shunts, wound drain tubes, skin sutures, vascular grafts and implantable meshes, intraocular devices, implantable drug delivery systems and heart valves, and the like. Alternatively, “prosthetic medical devices” may include, but are not limited to, artificial arms, artificial legs, or artificial hands.

The term “command signal” as used herein, refers to an extracted combination of neural signal indices which codes for a specific mental intention. For example, the command signal may provide instructions to (i.e., for example, “controlling”) move a natural appendage including but not limited to a leg, an arm, or a hand. Alternatively, the command signal may provide instructions to move a prosthetic medical device or activate a therapeutic medical device to release a therapeutic drug and/or initial deep brain stimulation.

The term “voluntary movement intention” as used herein, refers to y set of neural data signals generated by the conscious thought of a patient.

The term “involuntary movement intention” as used herein, refers to a set of neural data signals generated by unconscious thought of a patient.

The term “epileptic foci” as used herein, refers to a brain region responsible for the generation of an epileptic seizure as a result of aberrant neuronal action potential generation.

The term “dopamine-depleted neurons” as used herein, refers to a neuron that comprises less than normal levels of dopamine. Such neurons are generally thought to result in motor disorders that exhibit Parkinson's-like symptoms.

The term “drug” or “compound” as used herein, refers to a pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to a method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository, etc.

The term “at risk for” as used herein, refers to a medical condition or set of medical conditions exhibited by a patient, which may predispose the patient to a particular disease or affliction. For example, these conditions may result from influences that include, but are not limited to, behavioral, emotional, chemical, biochemical, or environmental influences.

The term “symptom”, as used herein, refers to subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but is not limited to, body temperature, complete blood count, lipid panels, thyroid panels, blood pressure, heart rate, electrocardiogram, tissue and/or body imaging scans.

The term “disease”, as used herein, refers to an impairment of a normal state of a living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors

The terms “reduce,” “inhibit,” “diminish,” “suppress,” “decrease,” “prevent” and “grammatical equivalents” (including “lower,” “smaller,” etc.), as used herein in reference to the expression of a symptom in an untreated subject relative to a treated subject, refers to a quantity and/or magnitude of the symptoms in the treated subject being lower than in the untreated subject by any amount that is recognized as clinically relevant by a medically trained personnel. The quantity and/or magnitude of the symptoms in the treated subject can be at least 10% lower than, at least 25% lower than, at least 50% lower than, at least 75% lower than, and/or at least 90% lower than the quantity and/or magnitude of the symptoms in the untreated subject.

The term “derived from” as used herein, refers to a source of a compound or sequence. In one respect, the compound or sequence may be derived from an organism or particular species. In another respect, the compound or sequence may be derived from a larger complex or sequence.

The terms “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, refers to any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “in operable combination” as used herein, refers to a linkage of device components in such a manner that a first component is capable of sending electronic information to the second component. Such linkages may involve high density connections, wires, cables, and or wireless communication technology.

### Brain-Machine Interface (BMI)

### I. Conventional Brain Machine Interfaces

Brain-machine interface (BMI) technology, where thoughts are turned into actions not by the body, but by computers and other machines, involves the reading and/or processing of brain neuronal signals. Brain-machine interfaces (BMI) have been reported to comprise arrays of hundreds of electrodes to sample the activities of multiple brain cells, from all over the brain, that are involved in the generation of movement. The electrical signals from the electrodes implanted in the brain were then sent to a computer, which learned how to extract the raw information. These methods decoded and translated the neuronal signals into a digital code representing the raw information that's embedded in the brain activity. The output of these models can then be used to control a variety of devices, such as robotic arms, wheelchairs or computer cursors, locally or remotely. Nicolelis, M., “Bionics: The Brain-Machine Interface” The Observer Health Magazine (Jul. 13, 2008).

Neuroscientists have long pondered the possibilities of using brain signals to control artificial devices. Schmidt E. M., Ann. Biomed. Eng. 8:339-349 (1980). As a consequence, there are already many terms in the literature to describe devices that could accomplish this goal (i.e., for example, brain-actuated technology, neuroprostheses and/or neurorobots, etc.). In: Chapin, J. K. & Moxon, K. A. (eds), Neural Prostheses for Restoration of Sensory and Motor Function (CRC, Boca Raton, 2000). The art has generally accepted terms such as ‘brain-machine interfaces” (BMI) or “hybrid brain-machine interfaces” (HBMIs) and are used interchangeably herein. The word ‘hybrid’ reflects the fact that these devices comprise continuous interactions between living brain tissue and artificial electronic or mechanical devices.

One type of BMI device uses artificially generated electrical signals to stimulate brain tissue in order to transmit some particular type of sensory information or to mimic a particular neurological function (i.e., for example, an auditory prosthesis). Future applications aimed at restoring other sensory functions, such as vision, by micro stimulation of specific brain areas would also belong to this group. In addition, type 1 HBMIs include methods for direct stimulation of the brain to alleviate pain, to control motor disorders such as Parkinson's disease, and to reduce epileptic activity by stimulation of cranial nerves. Benabid et al., Lancet 337:403-406 (1991); and Uthman et al., Epilepsia 31(Suppl. 2), S44-S50 (1990), respectively.

A second type of BMI device relies on real-time sampling and processing of large-scale brain activity to control artificial devices. An example of this application would be the use of neural signals derived from the motor cortex to control the movements of a prosthetic robotic arm in real time. Clinical applications comprising a reciprocal interaction between the brain and artificial devices would be expected to combine both HBMI types. The design and implementation of future HBMIs will involve the combined efforts of many areas of research, such as neuroscience, computer science, biomedical engineering, very large scale integration (VLSI) design and robotics.

Any HBMI development is founded upon an understanding of how neural ensembles encode sensory, motor and cognitive information. For example, primate motor control is fairly well studied, and considerable information is available on the physiological properties of individual neurons. On the other hand, little is understood as to how the brain makes use of neuronal signals to generate movements.

A. Recording Brain Activity

Primate studies have demonstrated that motor control emerges by the collective activation of large distributed populations of neurons in the primary motor cortex (M1). For example, single M1 neurons are believed to be broadly tuned to the direction of force required to generate a reaching arm movement. Georgopoulos et al., Science 233:1416-1419 (1986). In other words, even though these neurons fire maximally before the execution of a movement in one direction, they also fire significantly before the onset of arm movements in a broad range of other directions. Therefore, to compute a precise direction of arm movement, the brain may have to perform the equivalent of a neuronal ‘vote’ or, in mathematical terms, a vector summation of the activity of these broadly tuned neurons.

This implies that to obtain the motor signals to control an artificial device, the activity of many neurons should be monitored simultaneously and algorithms designed that are capable of extracting motor control signals from these ensembles. Moreover, different motor behaviors should be investigated to ascertain how these neural ensembles interact under more complex and ‘real-world’ experimental conditions. Ghazanfar et al., Trends Cog. Sci. 3:377-384 (1999).

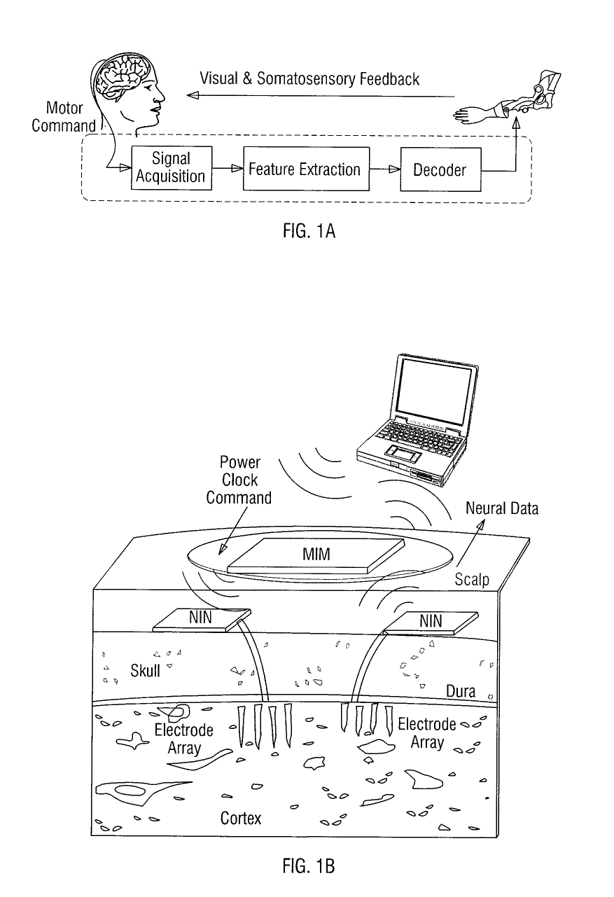

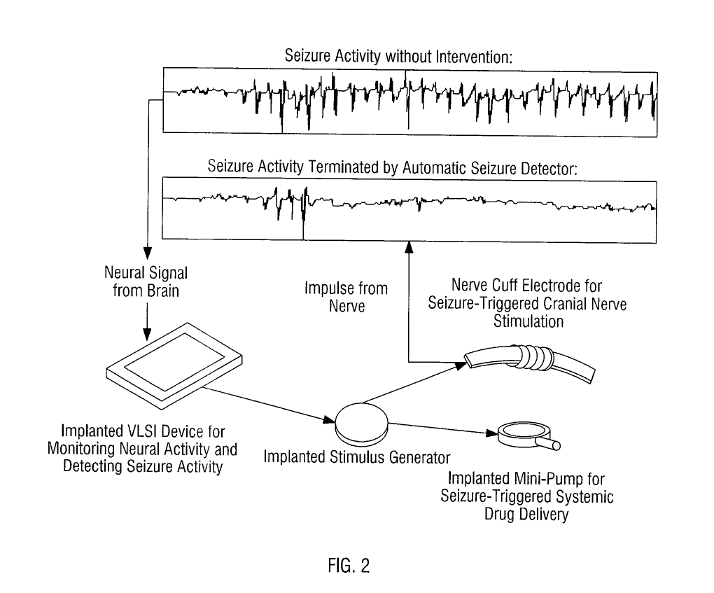

The general organization of a BMI system has numerous technological challenges involved in designing such devices. For example, a technique should be selected that yields reliable, stable and long-term recordings of brain activity that can be used as control signals to drive an artificial device. See, FIG. 1A. From recent animal studies, clinical applications of HBMIs will probably result in sampling of large numbers of neurons (i.e., for example, in the order of hundreds or thousands) with a temporal resolution of 10-100 ms, depending on the application. Chapin et al., L. Nature Neurosci. 2:664-670 (1999); and Wessberg et al., Nature 408:361-365 (2000).

Although there has been a long recognized need to investigate the properties of large neural ensembles, it is very difficult to obtain reliable, long-term measurements of neural ensemble activity with high spatial and temporal resolution. Hebb, D. O. “The Organization of Behaviour” In: A Neuropsychological Theory (Wiley, New York, 1949). For example, multichannel recordings of scalp electroencephalographic (EEG) activity and of the general electrical activity evoked by movement or sensory stimulation, a variety of metabolic, optical and electrophysiological methods have long been used for monitoring large-scale brain activity. Modern multichannel electrophysiological recordings are made from arrays of microelectrodes surgically implanted in the brain and allow simultaneous recording of up to 100 individual neurons with a resolution of milliseconds. Nicolelis et al., Nature Neurosci. 1:621-630 (1998). Although future improvements might allow long-term and non-invasive sampling of human neural activity with the same temporal resolution as intracranial recordings, first generation HBMIs are designed using these, electrophysiological methods. For example, EEG signals from paralyzed patients can control the movement of computer cursors or otherwise elicit communication. Wolpaw et al., Electroencephalogr. Clin. Neurophysiol. 78:252-259 (1991); and Schutz et al., Nature 398:297-298 (1999), respectively.

In general, these less invasive electrophysiological methods, have significant disadvantages in that they reflect the common electrical activity of millions of neurons in widespread areas of the brain and lack the resolution to provide the kind of time-varying input signals needed for specifically targeted performance (i.e., for example, fine muscle control). Multichannel intracranial recordings of brain activity, obtained by surgical implantation of arrays of microwires within one or more cortical motor areas is one approach that could result in a mathematical analysis of the extracellular activity of smaller populations (100-1,000) of neurons providing the raw brain signals for use in most HBMIs. Wessberg et al., Nature 408:361-365 (2000). Nonetheless, some degree of recording degradation is observed over time in the present technologies that allow simultaneous sampling of 50-100 neurons, distributed across multiple cortical areas of small primates, and thereby only remain viable for several years. Nicolelis et al., Nature Neurosci. 1:621-630 (1998).

A localized placement of electrode arrays for intracranial recording may be sufficient to control an artificial device because it has been observed that motor control signal emergence from the distributed activation of large populations of neurons may induce considerable cortical and subcortical neuronal plastic reorganization. Wu et al., J. Neurosci. 19:7679-7697 (1999). For example, as subjects learn to interact with artificial devices through HBMIs, it is likely that sampled neurons that were not originally involved in the type of motor control to be mimicked may be recruited into generating the signals required to control artificial devices.

B. Generating the Output

After selecting a BMI method for acquiring the brain signals, the next challenge is to design an instrument to record and/or process real time signals. See, FIGS. 1B-1D. Currently, these instruments are specialized, sizeable and expensive. For the most part, these instruments amplify and filter the original signals as well as perform analog-to-digital conversion to facilitate further processing and storage of data. To make implantable HBMIs viable, new technologies for portable, wireless-based, multichannel neural signal instrumentation are needed.

One approach to solving the problems of signal conditioning may utilize a mixed-signal VLSI in neurophysiological instrumentation chips. VLSI allows analog and digital signals to coexist in the same microchip, and has the potential to provide a multichannel, programmable and low-noise package required for conditioning brain-derived signals. Moreover, the resulting microchip would be small enough to be chronically implanted in patients and could be powered by replaceable batteries. Such microchips could rely on wireless communication protocols based on a radio frequency link to broadcast neural signals to other components of the HBMI. See, FIGS. 1D and 1E.

Dedicated ‘instrumentation neurochips’ are currently available, although many disadvantages must be overcome before they can become clinically useful. For example, efficient power supplies are not presently available to performing analog and digital processing, and still ensure that the conditioned signals can be wirelessly transmitted (i.e., for example, by telemetry). Thus, battery technology, device packing and the bandwidth of the neural signals, among other factors, are among the necessary improvements. Moxon et al., In: Neural Prostheses for Restoration of Sensory and Motor Function. (eds Chapin, J. K. & Moxon, K. A.) (CRC, Boca Raton, 2000).

Meaningful real time control information may also be extracted from neural ensemble activity. Currently, there exist a variety of linear and nonlinear multivariate algorithms, such as discriminant analysis, multiple linear regression and artificial neural networks, to carry out real-time and off-line analysis of neural ensemble data. Preliminary results from animal studies that use these different methods are useful, but considerable improvement is needed to apply these techniques in clinical HBMIs. The challenge is to produce algorithms that can combine the activity of large numbers of neurons, which convey different amounts of information, and extract stable control signals, even when the firing patterns of these neurons change significantly across different timescales. Research on areas ranging from automatic sorting algorithms for unsupervised isolation of single neuron action potentials, to the design of real-time pattern recognition algorithms that can handle data from thousands of simultaneously recorded neurons is currently lacking. In the same context, clinical applications of HBMIs will require considerable computational resources.

VLSI facilitates modeling neuronal systems in silicon, and may provide HBMIs with an efficient real time neural signal analysis. Hahnldser et al., Nature 405:947-951 (2000); and Mead C., In: Analog VLSI and Neural Systems (Addison-Wesley, Reading, Mass., 1989). VLSI may allow pattern recognition algorithms, such as artificial neural networks or realistic models of neural circuits, to be implemented directly in silicon circuits. Among many other technical hurdles, significant work will be required to make these silicon circuits adaptive, perhaps by incorporating learning rules derived from the study of biological neural circuits. This will allow ‘training’ of algorithms as well as ensuring the robustness of the control system. From an implementation point of view, ‘analytical neurochips’ are ideal as they could be interfaced with the instrumentation neurochip and be chronically implanted in the subject.

Real-time control interfaces which uses processed brain signals may be used to control an artificial device. The types of devices used are likely to vary considerably in each application, ranging from elaborate electrical pattern generators to control muscles, to complex robotic and computational devices designed to augment motor skills. Srinivasan, M. A., In: In Virtual Reality: Scientific and Technical Challenges (eds Durlach, N. I. & Mavour, A. S.) 161-187 (National Academy Press, 1994).

C. Output BMIs

A major goal of an ‘output BMI’ is to provide a command signal from a brain region (i.e., for example, the cortex). This command may serve as a functional output to control disabled body parts or physical devices, such as computers or robotic limbs. Finding a communication link emanating from the brain has been hindered by the lack of an adequate physical neural interface, by technological limitations in processing large amounts of data, and by the need to identify and implement mathematical tools that can convert complex neural signals into a useful command. BMIs that use neural signals from outside the cortex ('indirect BMIs') have already been developed for humans, and more recent efforts have produced ‘direct BMIs’ that use neural signals recorded from neurons within the cortex. Donoghue J. E., “Connecting cortex to machines: recent advances in brain interfaces” Nature Neuroscience Supplement 5:1085-1088 (2002).

1. Indirect BMIs

Indirect BMIs utilize a neural interface and report brain activity using a non-invasive procedure. For example, standard EEG electrodes noninvasively record electrical signals, which form the basis of several indirect BMIs. Other, existing indirect BMIs use scalp recordings which reflect the massed activity of many neurons. Signal quality may be improved with more invasive recordings where similar electrodes are placed on the dura or on the cortical surface. Various brain signals are being used as command sources. Individuals can learn to modulate slow cortical potentials (on the 0.5-10 time scale), adjust mu/beta EEG rhythms or use P300 as control signals. These signals can be readily acquired, averaged and discriminated with standard computers, which serve as the decoding instrument. In current devices, the command output is displayed on a computer screen, which serves as the machine component of the BMI and translates intent into a desired action. See, FIG. 4. Such systems can be successfully used by paralyzed humans to move a cursor on a computer screen or to indicate discrete choices. Wolpaw et al., “Brain-computer interfaces for communication and control” Clin. Neurophysiol. 113:767-791 (2001).

FIG. 4 presents a BMI according to one example embodiment. In the output BMI, neural interface detects the neurally coded intent, which is processed and decoded into movement command. The command drives physical device (computer) body part (paralyzed limb) that the intent becomes action. For input, stimulus is detected by physical device, coded into appropriate signal and then delivered by its interface the elicit percept (such touch vision). One of these inputs and outputs is determined by the individual through the voluntary interplay between percept and desired action.

Although current indirect BMIs can provide a functional output channel for paralyzed individuals, they still have many disadvantages. In particular, they are cumbersome to attach and are very slow compared to natural behavior. For example, multielectrode EEG systems can take an hour to configure and typically allow only a few output choices per minute. The output signal often depends on repeated samples, although changes in EEG frequency can provide some degree of real-time computer cursor control. The slowness of the system emerges from the indirect nature of the signals and the relatively long time (i.e., for example, several seconds) it takes for the user to modify those signals. It is relatively impossible for these BMIs to obtain a direct readout of movement intent because neural spiking that carries this information is lost by averaging and filtering across the scalp. Thus, the EEG signal used in indirect BMIs is a mere substitute for the actual neural signal that encodes actual movement. To be useful, the patient must therefore learn how to relate this arbitrary signal to an intended action, and because the signal is attention-related, use of the indirect BMI can interfere with other activities and control can be degraded by distractors.

2. Direct BMIs

Direct BMIs are intracortical recording devices designed to capture individual neuronal action potentials. In particular, those neuronal action potentials that code for movement or its intent. In comparison to indirect BMIs, direct BMIs are designed with a more demanding neural interface, more sophisticated signal processing, and more computationally intensive algorithms to decode neural activity into command signals. Direct BMIs are usually configured with microelectrode tips that are placed in close proximity to an individual neuron in order to gain access to their respective action potentials. To obtain a successful signal, electrodes must remain stable for long periods, and/or robust algorithms must be identified to deal with shifting populations. Some efforts have recorded a more degenerate signal from local field potentials, but this signal may be considerably limited in its information content in comparison to action potentials. Pesaran et al., “Temporal structure in neuronal activity during working memory in macaque parietal cortex” Nat. Neurosci. 5:805-811 (2002); Donoghue et al., “Neural discharge and local field potential oscillations in primate motor cortex during voluntary movements” J. Neurophysiol. 79: 159-173 (1998), respectively. Furthermore, the nature of information coding in the cortex has the added challenge of recording from many neurons simultaneously, especially if higher-order commands and high signal fidelity are desired. Reliable chronic multielectrode recording methods for the cerebral neocortex are at relatively early stages of development.

Several technologies have been suggested to support recordings from tens to hundreds of neurons that are stable for a period of months. Such assemblies are usually constructed of small wires, termed ‘microwires’, have been used for many years for chronic cortical recordings. These designs have been limited to use as experimental tools to study cortical activity. Marg et al., “Indwelling multiple micro-electrodes in the brain” Elecrroencephalogr. Clin. Neurophysiol. 23:277-280 (1967); Moxon et al., In: Neural Prostheses for Restoration of Sensory and Motor Function (eds. Chapin, K. & Moxon, K. A.) 179-219 (CRC Press, Boca Raton, Fla., 2000); and Pabner, C. “A microwire technique for recording single in unrestrained animals” Brain Res. Bull. 3:285-289 (1978).

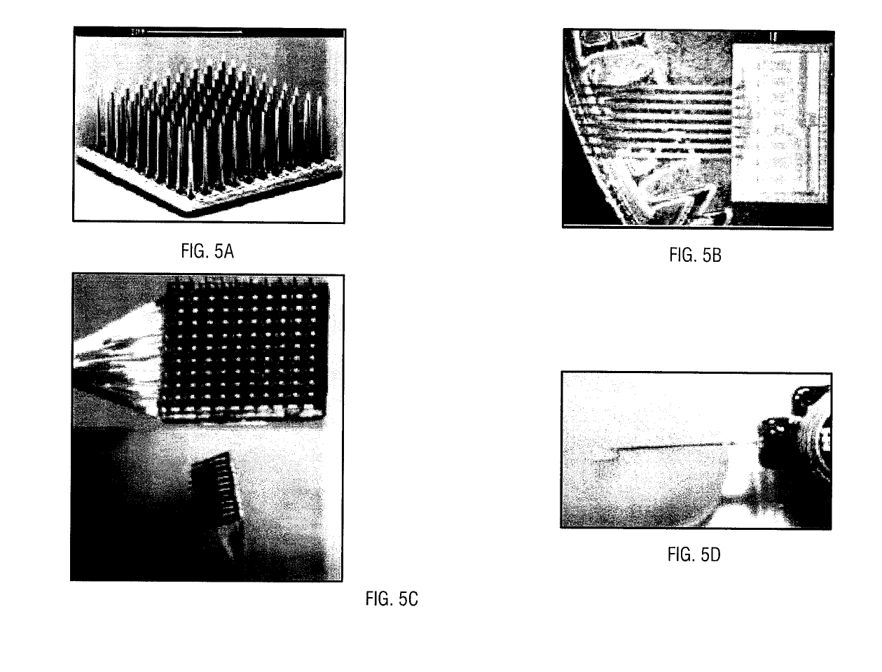

More advanced multiple electrode array systems are also being developed using advanced manufacturing and design methods, which is desirable for a reliable human medical device. See, FIG. 5. Bai et al., “Single-unit neural recording with active microelectrode arrays” IEEE Trans. Biomed. Eng. 48:911-920 (2001). These neural interfaces, plus microribbon cables, connectors, and telemetry devices have been shown to record multiple neurons in humans. Miniaturization techniques have allowed the placement of such devices within the confines of the skull, wherein small, high density connectors interconnect the components, and telemetry transmits the neuronal signals to remote processors or effectors. Maynard et al., “The Utah Intracortical Electrode Array: recording structure for potential brain-computer interfaces” Electroencephalogr. Clin. Neurophysiol. 102:228-239 (1997); Rousche et al, “Flexible polyimide-based intracortical electrode arrays with bioactive capability” IEEE Trans. Biomed. Eng. 48:361-371 (2001); and Nicolelis, M. A. L., “Actions from thoughts” Nature 409:403-407 (2001). Each of these components is under development, but they present formidable technical challenges.

Current arrays are nevertheless reasonable prototypes for a human BMI. They are relatively small in scale and some have been successfully used for chronic recording. For example, individual electrodes in the Utah electrode are tapered to a tip, with diameters<90 μm at their base, and they penetrate only 1-2 mm into the brain; these electrodes have been reported to support prolonged recording in monkey cortex. Maynard et al., “Neuronal interactions improve cortical population coding of movement direction” J. Neurosci. 19:8083-8093 (1999); and Serruya et al., “Instant neural control of movement signal” Nature 416:141-142 (2002). Intracortical arrays are on a microscale as compared to devices such as intraventricular catheters to treat hydrocephalus (i.e., for example, approximately 2-3 mm in diameter) or deep brain stimulator electrodes, which are now accepted as safe human brain implants. See, FIG. 5B.

Neurotrophic recording electrodes are also being tested as potential direct BMI devices. Kennedy et al., “Direct control of computer from the human central system” IEEE Trans Rehabil. Eng. 2:198-202 (2000). These electrodes, which have been used to record from human motor cortex, are small glass cones inserted individually into the motor cortex; each cone contains recording wires and factors that induce neural process ingrowth. These technologies may be the most advanced candidates for a direct human cortical interface. Devices that detect action potentials without displacing neural tissue are highly desirable, but no such method is available.

After recording neural signals, signal conditioning/processing is used to isolate a useful command signal. Multiple neuron recordings provide a significantly more challenging decoding problem than EEG signals, both because the signal is complex and because of large input processing demands. First, electrical activity is digitized at high rates (>20 kHz) for many channels, action potentials must be sorted from noise, and decoding algorithms must process neural activity into a useful command signal within a meaningful time frame, all on the order of 200 ms. A further challenge is to extract a command signal that represents movement intent. A vast body of literature documents that populations of neurons carry considerable information about movement commands. Neural firing rate or pattern in motor areas carries sensory, motor, perceptual and cognitive information. Pioneering work has demonstrated that motor cortical neurons can provide reliable estimates of motor intentions, including force and direction. Homphrey et al., “Predicting of motor performance from multiple cortical spike trains” Science 170:758-762 (1970); and Georgopoulos, A. E., “Population activity in the control of movement” Int. Rev. Neurobiol 37:103-119 (1994).

Recently however, three groups have demonstrated that hand trajectory can be recovered from the activity of populations of neurons in motor cortex. Serruya et al., “Instant neural control of movement signal” Nature 416:141-142 (2002); Taylor et al., “Direct cortical control of 3D neuroprosthetic devices” Science 296:1829-1832 (2002); and Wessberg et al., “Real-time prediction of hand trajectory by ensembles of cortical in primates” Nature 408:361-365 (2000). These same groups also developed mathematical methods and took advantage of technological enhancements to demonstrate real-time reconstruction of monkey hand motion as it unfolds in a reaching task.

Mathematical decoding methods, such as linear regression, population vector and neural network models, have shown that the firing rate of motor cortex populations provides an estimate of how the hand is moving through space. Advances in modeling have resulted in the discovery that brain output connected to robot arms or computer cursors can mimic a monkey's ongoing arm movements, showing that neural decoding is fast and accurate enough to be a spatial control command. Ongoing efforts in mathematical decoding suggest that both the quality and form of movement reconstructions may be further improved when interactions among neurons or additional signal features are considered. Maynard et al., “Neuronal interactions improve cortical population coding of movement direction” J. Neurosci. 19:8083-8093 (1999); and Gao et al., “Probabilistic inference of hand motion from neural activity in motor cortex” Proc. Adv Neural Info. Processing Systems 14, The MIT Press (2002). Nonetheless, these signals are far from providing the full repertoire of movements that the arm can produce, such as manipulative movements of the fingers or grip control. Moreover, dealing with more complex actions or the simultaneous control of multiple, independent body parts will likely require more electrodes and more arrays.

3. Cortical Control of BMIs

As discussed above, recent work has shown that cortically derived command signals can substitute for hand motion in behavioral tasks. Monkeys were able to move a cursor to targets displayed on a computer monitor solely by brain output where neural control of the cursor could continue whether or not the original tracking hand motions were present. There is no direct evidence suggesting that the monkeys understood that the brain directly controlled the cursor, but one cannot fully rule out the possibility that the monkey learned some covert action to achieve cursor control. There has been great interest in knowing whether humans might be able to gain direct control over their own neurons, both from its fascinating implications and from a practical perspective for paralyzed patients. This question can be more readily resolved by recording in paralyzed humans, where it has been specifically addressed.

For example, voluntarily generated neural activity in the motor cortex of a patient with near-total paralysis has been demonstrated. Kennedy et al., “Direct control of computer from the human central system” IEEE Trans Rehabil. Eng. 2:198-202 (2000). Using activity obtained through a few channels from implanted cone electrodes, the patient was able to move a cursor on a computer screen. So far, the level of control using the cone electrode has not matched that seen in monkeys; human control has been slower and with more limited dimensionality, on par with that seen in the indirect BMIs. The reasons for this discrepancy are not clear.

D. Input BMIs

Converting motor intent to a command output signal can restore the ability to act upon the environment. However, sensory input is also involved in controlling normal interactions, especially when outcomes of behavior are unreliable or unpredictable. An ideal communication interface for patients lacking intact somatic sensory pathways would be able to deliver signals to the cortex that are indistinguishable from a natural stimulus.

Two recent findings indicate the potential to return meaningful information to the cortex by using local electrical microstimulation within the cortex. For example, microstimulation of the somatic sensory cortex can substitute for skin vibration in a perceptual task requiring frequency discrimination based on either skin or electrical stimulation. Romo et al., “Sensing without touching: psychophysical performance based cortical microstimulation” Neuron 26:273-278 (2000). Similarly, rats can use electrical stimulation to their cortical whisker areas as a directional cue for left-right motions. Talwar et al., “Rat navigation guided by remote control. Nature 417:37-38 (2002). These findings are supported by other studies suggesting that it will be possible to construct stimulation patterns that humans can use in a meaningful way to form percepts when natural systems are not available. Wickersham et al., “Neurophysiology: electrically evoking sensory experience” Curr Biol. 8:R412-R414 (1998).

There is a difference between these types of electrical stimulation, (which are intended to replace the natural percept) and other forms of stimulation which have attempted to drive behavior or modify brain function without the recipient's cognitive intervention. Delgado, J. M. Physical Control of the Mind (Harper and Rowe, New York, 1969). Cortical input BMIs may also be applied to other forms of sensory loss. Of particular interest is the visual prosthesis designed to restore sight by direct stimulation of the visual cortex. Both cortical surface and intracortical stimulation have been shown to generate phosphenes, although considerable research is needed to understand how to move from spots of light to restoration of useful images of the world. Dobelle, W. H., “Artificial vision for the blind by connecting television to the visual” ASAIO J. 46:3-9 (2000); Hambrecht, E T., “Visual prostheses based direct interfaces with the visual system” Baillieres Clin. Neurol 4:147-165 (1995); Maynard, E. M., “Visual prostheses” Annu. Rev. Biomed. Eng. 3:145-168 (2001); Normann et al., “A neural interface for cortical vision prosthesis” Vision Res. 39:2577-2587 (1999); Schmidt et al., “Feasibility of visual prosthesis for the blind based intracortical micro stimulation of the visual cortex” Brain 119: 507-522 (1996).

### II. Intra-Cortical Neural Interface Systems

In one example embodiment, a system comprising devices and real time methods for acquiring, transmitting, and processing neural signals from a brain is provided. In one embodiment, the brain comprises a plurality of interconnected neuronal cells. In one embodiment, the brain comprises an individual neuronal cell. In one embodiment, the system further comprises a plurality of devices comprising integrated microchips. In one embodiment, at least one of the devices comprises a brain-machine interface device. In one embodiment, at least one of the devices comprises a data transmission device. In one embodiment, at least one of the devices comprises a data storage device. In one embodiment, the microchips comprise a plurality of sensors, wherein the sensors are deployed as large scale integrated circuits. In one embodiment, the acquiring is continuous. In one embodiment, the transmitting is wireless.

Electronic data transmitter components are widely available wherein a device compatible with the above described system may be constructed from commercially available components. On the other hand, the implanted microchip is much more complex and requires not only, novel circuitry designs but also novel algorithms to process the large bandwidth neuronal data stream. Microchip-algorithm development is an empirical process with regular testing of subcomponents to ensure their overall compatibility with the system. For example, once a prototype algorithm-chip is constructed, an animal experiment is performed to implant the prototype and collect data from an immobilized, awake animal. The data collected from these empirical tests are compared against commercial data acquisition systems to ensure data reliability. Once a preferred prototype algorithm-chip has been optimized, the chip will be implanted and testing in an unrestricted (i.e., untethered) environment. This testing should identify artifacts that may interfere with signal processing and/or wireless transmission. Other animal studies compare signals acquired related to specific behavior using a wired and wireless system.

In one example embodiment, t a method comprising a real time telemetry-based BMI system including, but not limited to: i) amplifying and filtering of an analog signal (i.e., for example, neural voltage waveforms ranging between approximately 100-900 microvolts); ii) conversion of the analog signal into a digital signal compatible with known storage and transmission systems; iii) transforming the digital signals to a new analysis domain (i.e., for example, a wavelet domain transform, DWT); iv) thresholding the transformed signals for denoising, signal detection and classification; v) processing digital signal to extract neuronal data signal information; vi) compressing the threshold signals for wireless telemetry; vi) formatting the compressed data for short range communication (a few mm's) through a NIN module (i.e., for example, an implanted microchip); vii) receiving the compressed data at a MIM module (i.e., for example, a transmitter) and extracting additional biological information; viii) formatting the extracted data for long range communication to a central base station for further processing, decoding, and control; and iv) create output information for use by neuroscientists, is provided. In one embodiment, the processing capability is compatible with clinical constraints comprising low power, small size and/or wireless connectivity.

Currently, neuronal data signal information is usually extracted using a standalone microprocessor unit (i.e., for example, a desktop computer). In one embodiment, an implantable microchip comprising a plurality of microelectrodes is provided. In one embodiment, the microchip further comprises an algorithm for extracting neuronal data signal information. In one embodiment, the microchip is connected to a transmitter. Although not wishing to be bound by this proposed theory, it is believed that one advantage of the embodiments described herein is that the wireless data transmission system may use smart signal processing to extract the information prior to transmission. Smart signal processing minimizes bandwidth, thereby overcoming conventional wireless transmission constraints.

The art has found numerous barriers to the successful development of wireless neuronal data streaming that reside primarily in microchip design. Nonetheless, some example embodiments take advantage of microchip designs by envisioning a modular architecture. For example, many brain regions may be processed and analyzed simultaneously using a system comprising a flexible channel capability. In one embodiment, the system may process and analyze thirty-two channels. In one embodiment, the system may process and analyzed sixty-four channels. In other embodiments, the microchips can be designed to process qualitatively different information collected simultaneously, or serially, from a single neuronal cell and/or a plurality of neuronal cells that represent an interconnected neural network. This type of modular architecture means that the systems described herein are not restricted by the specific signal modality or desired application or a specific electrode design.

Many conventional BMI and/or HBMI systems discussed herein have numerous disadvantages that are discussed herein. In some example embodiments, a system is provided which has advantages which include, but are not limited to: a) high capacity, suited for large scale interfaces with the nervous system; 2) Real time Signal Processing capability; 3) Fully wireless to minimize any potential risk of infection and discomfort to the patient in clinical settings; 4) Preserves all the desired information in the recorded neural signals; 5) Highly modular to allow scalability to arbitrary sizes to suit a wide variety of animal models and/or human clinical applications; 6) Adaptive to changes in neural signals in long term experiments/clinical use; 7) Versatile, reliable and programmable for bi-directional communication; and/or 8) State of the art signal processing technology for “smart” information extraction decreases the necessary bandwidth and allows for a fully wireless system.

### III. Compressive Spike Sorting Algorithms

In one example embodiment, an algorithm for sorting neural spikes is provided. In one embodiment, the neural spike comprises a plurality of action potentials. In one embodiment, the plurality of action potentials are derived from multiple nerve cells (i.e., for example, neurons). In one embodiment, the plurality of action potentials is derived from a single neuron. In one embodiment, the plurality of action potentials is simultaneously recorded. In one embodiment, the plurality of action potentials is recorded using a single microelectrode. In one embodiment, the plurality of action potentials is recorded using an array of microelectrodes. In one embodiment, the algorithm resides on an implantable microchip. In one embodiment, the microchip is ultra low power. In one embodiment, the microchip comprises miniaturized electronic circuits.

Many disadvantages exist in regards to current technology that support neurophysiology data acquisition systems including, but not limited to, being bulky, hard wired, very expensive, and requiring large computational power to support spike sorting. Many of the current systems require high electrode channel count and large number of cells to operate efficiently and reliably. In some embodiments, the neural spike sorting algorithm solves many of these disadvantages that facilitate the development of fully implantable, practical, and clinically viable brain machine interfaces. These advantages of the presently contemplated according to some example embodiments include, but are not limited to: 1) classifying multiple spike waveforms (i.e., for example, spike sorting) to permit extracting spike trains of individual neurons from the recorded mixture of signals; 2) reducing the ultra-high communication bandwidth needed to transmit the recorded raw data and permit offline waveform classification of these waveforms; 3) extracting information reliably from single cell activity to characterize brain function in normal healthy individuals and also in subjects suffering from many neurological diseases and disorders including, but not limited to, Parkinson's disease and/or epilepsy; or 4) improving assistive technology to treat severe paralysis or impaired movements from spinal cord injury by directly translating the neural signals monitored in the brain that are related to movement intention to control commands that operate prosthetic limbs.

Neuronal spike trains comprise a neural communication mechanism used by cortical neurons to relay, process, and store information in the central nervous system. Decoding the information in these spike trains would be expected to reveal the complex mechanisms underlying brain function. For example, in motor systems, these spike trains were demonstrated to carry important information about movement intention and execution. Georgopoulos et al., “Neuronal population coding of movement direction” Science 233:1416 (1986). Further, these motor spike trains were shown to be useful in the development of neuroprosthetic devices and brain-machine interface (BMI) technology to assist people suffering from severe disability in improving their lifestyle. Hochberg et al., “Neuronal ensemble control of prosthetic devices by a human with tetraplegia,” Nature 442:164-171 (2006); and Taylor et al., “Direct cortical control of 3D neuroprosthetic devices” Science 296: 1829 (2002).

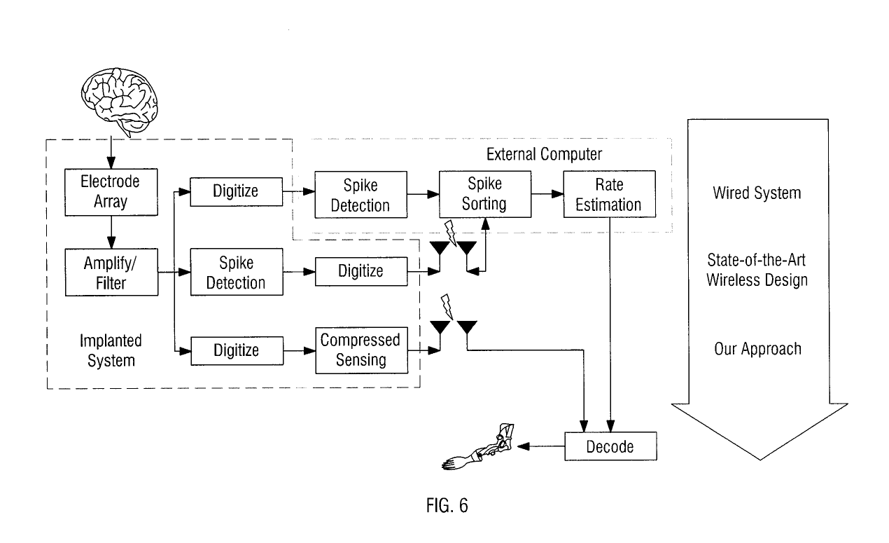

Currently available cortically-controlled BMI systems may instantaneously decode spike trains from motor cortical neurons recorded during a very limited interval. This limited interval, often referred to as the movement planning period, is estimated to be approximately 100-200 milliseconds (ms). Moran et al., “Motor cortical representation of speed and direction during reaching” J. Neurophysiol. 82:2676-2692 (1999). Decoding processes are typically a cascade of data processing steps. See, FIG. 6.

FIG. 6 presents that ensemble neural recordings are first amplified and filtered prior to telemetry transmission to the outside world. Three data processing paths are considered. 1) Wired systems (top): information is extracted through the cascade of spike detection and sorting followed by rate estimation with a massive computational power. Hochberg et al., “Neuronal ensemble control of prosthetic devices by a human with tetraplegia” Nature 442:164-171 (2006). 2) Wireless systems (middle): Telemetry bandwidth is reduced by moving the spike detection block inside the implantable device. Harrison et al., “A low-power integrated circuit for a wireless 100-electrode neural recording system,” IEEE J. Solid State Circ. 42:123-133 (2007); and Wise et al., “Microelectrodes, microelectronics, and implantable neural microsystem,” Proc. IEEE 96:1184-1202 (2008). 3) Proposed system (bottom): the spike detection, sorting and rate estimation blocks are replaced with one “compressed sensing” block that permits adaptive firing rate estimation in real time for instantaneous decoding to take place.

Decoding processing generally features amplifying and filtering, followed by detecting spikes and sorting the spikes to segregate single unit responses in the form of binary spike trains. The spike trains may then be filtered using, for example, a variable-width kernel function (e.g., a Gaussian) to yield a smoothed estimate of the instantaneous firing rate. Kass et al., “Statistical smoothing of neuronal data,” Network Computat. Neural Syst. 14:5-15 (2003); and Paulin et al., “Optimal firing rate estimation” Neural Networks 14:877-881 (2001). Although not wishing to be bound by this proposed theory, it is believed these steps are performed within a movement preparation period to enable the subject to experience a natural motor behavior.

Spike sorting has always represented the most computationally challenging in the processing sequence. In general, spike sorting involves at least two modes of analysis: a training mode and a runtime mode. During the training mode, spikes are detected, aligned, and sorted based on certain discriminating features, such as principal component analysis (PCA) scores. Lewicki M., “A review of methods for spike sorting: The detection and classification of neural action potentials” Network: Computat. Neural Syst. 9:53-78 (1998). During runtime, an observed spike's features are compared to the stored features to determine which neuronal class it belongs to. Both steps involve a significant amount of computations to enable this identification/classification process to run smoothly. As a result, most existing systems feature a wired connection to the brain to permit streaming the high-bandwidth neural data to the outside world where relatively unlimited computing power can carry out this task with close to real time performance.

Alternative processing methods for neural data have proposed; i) denoising and compression (Oweiss, K., “A systems approach for data compression and latency reduction in cortically controlled brain machine interfaces” IEEE Trans. Biomed. Eng. 53:1364-1377 (2006); ii) spike detection and sorting based on a sparse representation of the recorded data prior to telemetry transmission. (Oweiss, K., “Multiresolution analysis of multichannel neural recordings in the context of signal detection, estimation, classification and noise suppression,” Ph.D. dissertation, Univ. Michigan, Ann Arbor, 2002; and Oweiss et al, “Tracking signal subspace invariance for blind separation and classification of nonorthogonal sources in correlated noise” EURASIP J. Adv. Signal Process 2007:20 (2007). Further reports discuss the suitability of such processing systems to support a wireless implantable system. Oweiss et al., “A scalable wavelet transform VLSI architecture for real-time signal processing in high-density intra-cortical implants,” IEEE Trans. Circuits Syst. 154:1266-1278 (2007). Recent improvements in signal processing have suggested methods to overcome the severe bandwidth limitations of a wireless implantable system, and provide an adequate estimation of neuronal firing rates without the need to use traditional methods to decompress, reconstruct, and sort the spikes ‘off-chip’. See, FIG. 6, bottom. These improved methods decode neural discharge patterns using only the compressed data.

A. Single Neuron Point Process Model

In a typical recording experiment, the observations of interest are the times of occurrence of events from a population of neurons and expressing the discharge pattern of these neurons. In an arbitrary neuron, the firing can be modeled as a realization of an underlying point process with a conditional intensity function and/or firing rate, λₚ (t F). Brown N., “Theory of point processes for neural systems,” In: Methods and Models in Neurophysics, C. C. Chow, Ed. et al. Paris, France: Elsevier, 2005, pp. 691-726. This intensity function is conditioned on some set, F, of intrinsic properties of the neuron itself and the neurons connected to it, and some extrinsic properties such as the neuron's tuning characteristics to external stimuli features during that trial. Because many of these properties are hard to measure, the number of events in a given interval, N_{P}, is typically random by nature. Consequently, the integral of λₚ over a finite time interval [Tₐ, T_{b}] represents the expected value within a single trial:

E[Nₚ]=∫_{T}_{_{c}}^{T}^{_{b}}λₚ(t|F)dt. (1)

Brillinger D., “Nerve cell spike train data analysis” J. Am. Stat. Assoc. 87:260-271 (1992). Estimating λₚ from the set of event times [tₚ] is typically achieved by binning the data into time bins of equal width, T_{w}=T_{b}−Tₐ, and counting the number of events occurring within each bin. The resulting spike counts, often referred to as a rate histogram, constitute an instantaneous firing rate estimate. In traditional signal processing, this is equivalent to convolving the spike train with a fixed-width rectangular window. This approach assumes that variations in the rate pattern over the bin width do not carry information that is destroyed if aliasing occurs, for example, when the bin width is not optimally selected to satisfy the Nyquist sampling rate of λₚ.

The binning approach can detect the presence of the type of spike bursts that may exist within the fixed-length bins. However, bursts come in a variety of lengths within a given trial, and can range from very short bursts (3-4 spikes within 2-3 ms to much longer bursts that can last for more than 2 s. Kaneoke et al., “Burst and oscillation as disparate neuronal properties,” J. Neurosci. Methods 68, pp. 211-223, 1996; and Goldberg et al., “Enhanced synchrony among primary motor cortex neurons in the 1-methyl-4-phenyl-1,2,3,6-tetrahydropyridine primate model of Parkinson's disease” J. Neurosci. 22:4639 (2002). This implies that the firing rate of individual neurons is highly nonstationary and that temporal and spectral variations in λₚ are believed to occur over a multitude of time scales that reflect the complex temporal structure of neuronal encoding while subjects carry out similar behavioial tasks or depending on the demands of distinct behavioral tasks. Churchland et al., “Temporal complexity and heterogeneity of single-neuron activity in premotor and motor cortex,” J. Neurophysiol. 97:4235 (2007); Shadlen et al., “The variable discharge of cortical neurons: Implications for connectivity, computation, and information coding” J. Neurosci. 18:3870-3896 (1998); and Kass et al., “Spike count correlation increases with length of time interval in the presence of trial-to-trial variation” Neural Computat., 18:2583 (2006), respectively. This “non-stationarity” arises in part because of the dependence of the firing rate on multiple factors such as the degree of tuning (sharp or broad) to behavioral parameters, the behavioral state, the subject's level of attention to the task, level of fatigue, prior experience with the task, etc. While across-trial averaging of rate histograms (peristimulus) helps to reduce this variability, it destroys any information about the dynamics of interaction between neurons that are widely believed to affect the receptive fields of cortical neurons, particularly when plastic changes occur across multiple repeated trials, typically a nonparametric kernel smoothing step (e.g., a Parzen window). Parzen, E., “On estimation of a probability density function and mode,” Ann. Math. Stat., 33:1065-1076 (1962). The temporal support T_{w} of the kernel function is known to strongly impact the rate estimator. Cherif et al., “An improved method for the estimation of firing rate dynamics using an optimal digital filter” J. Neurosci. Methods 173:165-181 (2008). Moreover, the selection of T_{w} is arguably important to determine the type of neural response property sought. For small T_{w} (i.e., for example, <2-3 ms), precise event times can be obtained. As T_{w} approaches the trial length, an overall average firing rate is obtained over that trial. In between these two limits, T_{w} needs to be adaptively selected to capture any nonstationarities in λₚ that may reflect continuously varying degrees of neuronal inhibition and excitation indicative of variable degree of tuning to behavioral parameters.

B. Sparse Extracellular Spike Recordings

λₚ may be estimated directly from the recorded raw data. However, the detected events are not directly manifested as binary sequence of zeros and ones to permit direct convolution with a kernel to take place, but rather by full action potential (AP) waveforms. Additionally, these events are typically a combination of multiple single unit activity in the form of AP waveforms with generally distinct-but occasionally similar-shapes. This mandates the spike sorting step before the actual firing rate can be estimated.

Assuming that the actual spike waveforms are uniformly sampled over a period T_{∂} Each spike from neuron p is a vector of length N₈ samples that Applicants will denote by gₚ. For simplicity assume the event time is taken as the first sample of the spike waveform (this can be generalized to any time index, e.g., that of a detection threshold crossing). The discrete time series corresponding to the entire activity of neuron p over a single trial of length T can be expressed as:

$${S}_{p} = {\sum }_{i ∈ \{ {t}_{p} \}} {\sum }_{k = 0}^{{N}_{s} - 1} {g}_{p} [ k ] δ [ i + k ] \qquad ( 2 )$$

where the time index i includes all the refractory and rebound effects of the neuron and takes values from the set {tₚ}, while δ(·) is the Dirac delta function. For compression purposes, it was shown that a carefully-chosen sparse transformation operator, such as a wavelet transform, can significantly reduce the number of coefficients representing each spike waveform to some N_{c}<<N_{∂}. Oweiss, K., “A systems approach for data compression and latency reduction in cortically controlled brain machine interfaces” IEEE Trans. Biomed. Eng. 53:1364-1377 (2006); and Oweiss, K., “Multiresolution analysis of multichannel neural recordings in the context of signal detection, estimation, classification and noise suppression,” Ph.D. dissertation, Univ. Michigan, Ann Arbor (2002). This number is determined based on the degree of sparseness q as N_{c}ε^{(q-2)/2q} as where 0<q<2 (q=0 implies no sparseness while q=2 implies fully sparse) and c denotes some arbitrarily chosen signal reconstruction error. Candes et al., “Robust uncertainty principles: Exact signal reconstruction from highly incomplete frequency information” IEEE Trans. Inf. Theory 52:489-509 (2006). Mathematically, an observed spike, g, is represented by the transform coefficients obtained from the inner product g^{j}=(g, wⱼ), where wⱼ is an arbitrary wavelet basis at time scale j. When multiple units are simultaneously recorded, the spike recordings from the entire population can be expressed as:

$$N \frac{\partial }{c} - 1$$

$${s}^{j} = {\sum }_{i ∈ \{ {t}_{s}^{j} \}} {\sum }_{k = 0}^{⋁} {g}^{j} [ k ] δ [ i + k ]$$

where

$$N \frac{\partial }{c} - 1$$

is the number of nonzero transform coefficients, and i takes values from the set of spike times for all neurons in the whole trial,

$$\{ t \frac{j}{s} \} .$$

Note that

$$N \frac{\partial }{c} - 1 ≪ {N}_{8}$$

and the total number of coefficients obtained is

$${N}_{c} = \sum N \frac{j}{\partial } .$$

To minimize the number of the most important a coefficients/event, ideally to a single feature, the magnitude of the coefficients g^{j} carry information about the degree of correlation of the spike waveforms with the basis wⱼ. Therefore, this information can be used to single out one feature out of “the most significant” coefficients (i.e., for example, create a discarded subset and/or a retained subset) per event from neuron via a thresholding process. One way to obtain this single feature, fgⁱ[k] is to locally average the coefficient before thresholding. In one example embodiment, Applicants define a neuron-specific sensing threshold at time scale jj, denoted

$$γ \frac{j}{p} .$$

This threshold is selected to preserve the ability to discriminate neuron p's events from those belonging to other neurons using this single feature. Specifically, in every time scale j, the problem may be cast as a binary hypothesis test in which:

$${f}_{g}^{j} [ k ] {{≷}_{{H}_{0}}}^{{H}_{1}} {γ}_{p}^{j} k = 0 , 1 , … , {N}_{c}^{j} , j = 0 , 1 , … , J . \qquad ( 4 )$$

Using a top-down approach,

$$γ \frac{j}{p}$$

is selected based on a standard likelihood ratio test (given predetermined level of false positive). The outcome of this statistical binary test is a one time index per event, k*, for which the alternative hypothesis H₁ is in effect. In other words, the sensing threshold in a given time scale may allow only one feature to be kept per event. Once this is achieved, fgⁱ[k] at indices where H₀ is in effect are automatically set to zero. Note that this step allows suppressing noise coefficients as well as those belonging to neurons' other than neuron p's. In such case, the threshold signal can be expressed as:

$${\overline{s}}_{p}^{j} = {\sum }_{i ∈ \{ {t}_{p}^{i} \}} {f}_{g}^{j} [ {k}^{*} ] δ [ i - {k}^{*} ] . \qquad ( 5 )$$

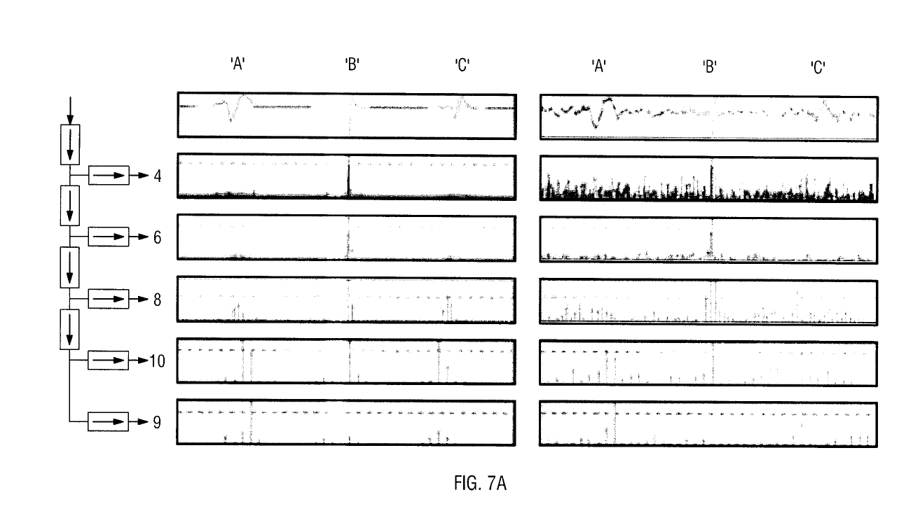

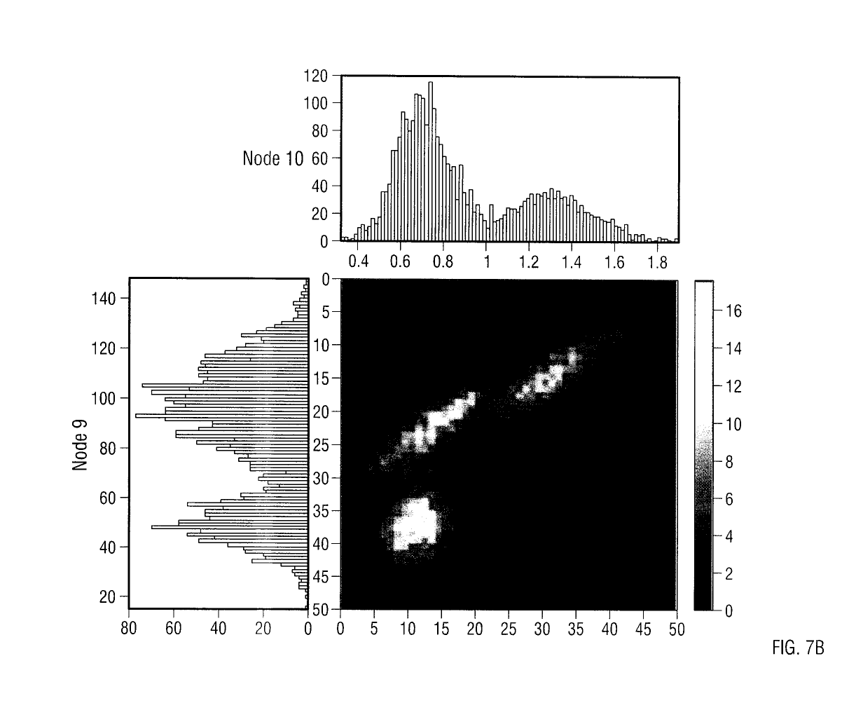

The outcome of equation (5), after proper normalization fg^{j}[k*], is an estimate of the true binary spike train vector. It can be readily seen that the temporal characteristics of this estimate will exactly match that of the binary spike train of neuron and consequently preserves information including, but not limited to, spike counts and interspike interval (ISI) statistics allowing rate estimation to be readily implemented. See, FIG. 7. Oweiss K., “Compressed and distributed sensing of multivariate neural point processes,” In: IEEE Int. Conf. Acoustics, Speech Signal Process., Apr. 15-20, 2007, vol. 2, pp. 577-580. In each wavelet decomposition level, the binary hypothesis test (i.e., the thresholding) is equivalent to a two-class discrimination task whereby one unit at a time is identified at each level. The spike class separability (defined below) is compared to that in the time domain and a unit is extracted (i.e., its coefficients removed) from the data set if the unit separability is higher than that of the time domain. This process is repeated until the separability no longer exceeds that of the time domain, or the size of the remaining events is smaller than a minimum cluster size (typically five events), or the maximum number of decomposition levels has been reached (typically 4-5 levels).

### C. Instantaneous rate Estimation

A fundamental property of the DWT sparse representation suggests that as j increases,

$$\hat{s} \frac{j}{p}$$

becomes more representative of the intensity function rather than the temporal details of neuron p's spikes, which were eventually captured in finer time scales. This is because the coefficients that survive the sensing threshold will spread their energy across multiple adjacent time indices, thereby performing the same role as the kernel smoothing approach, but at a much less computational overhead as will be shown later. Mathematically, extending the DWT of the vector

$$\hat{s} \frac{j}{p}$$

after normalization to higher level requires convolving it with a wavelet basis kernel with increasing support.

This support, denoted at level t_{L}, is related to the sampling period Tₛ by:

t_{L}=Tₛn_{w}2^{(L-2)} (6)

where n_{w} is the wavelet filter support. For the symmlet4 basis used herein (n_{w}=8), this temporal support is equivalent to ˜2 ms at level 4 (at 25 kHz sampling rate), which roughly corresponds to one full event duration. Extending the decomposition to level 5 will include refractory and rebound effects of neurons typically observed in the cerebral cortex. Churchland et al., “Temporal complexity and heterogeneity of single-neuron activity in premotor and motor cortex” J. Neurophysiol, 974235 (2007). Therefore, temporal characteristics of the firing rate will be best characterized starting at level 6 and beyond where the basis support becomes long enough to include two or more consecutive spike events

### A. Computational Complexity

Herein, Applicants compare the cost of estimating the firing rate through the standard time domain spike sorting/kernel smoothing approach and the proposed compressed sensing approach. Both involve calculating the computational cost in two different modes of operation, the “training” mode and the “runtime” mode. In the training mode, features are extracted and the population size is estimated using cluster cutting in the feature space. This may ideally correspond to the number of distinct spike templates in the data. In the runtime mode, the observed waveforms are assigned to any of the existing classes, typically using a Bayesian classifier with equal priors

$$\begin{matrix}p = {arg max}_{p} P ( {C}_{p} \vert g ) \\ = {arg max}_{p} \frac{P ( g \vert {C}_{p} )}{P ( {C}_{p} )} P ( g ) ⇒ p ≅ {arg max}_{p} P ( g \vert {C}_{p} )\end{matrix} \\ P ( g \vert {C}_{p} ) = \frac{1}{{( 2 π )}^{{N}_{s} / 2} {\vert {Σ}_{p} \vert}^{{N}_{s}}} exp [ - \frac{1}{2} {( g - {μ}_{p} )}^{T} {\sum }_{p}^{- 1} ( g - {μ}_{p} ) ] \qquad ( 7 )$$

where μₚ and Σₚ are the Nₛ×1 mean vector and Nₛ×Nₛ temporal covariance matrix for each neuron p=1, . . . , P. The overall computations for the Bayesian classifier are in the order of ˜O(Nₛ²P).

A standard PCA-based spike sorting followed by a Gaussian Kernel rate estimator was used as the benchmark for evaluating the computational cost of the traditional path that appears in the top of FIG. 6. First, spikes are aligned by searching for a local extreme followed by cropping the waveform symmetrically around that location, which requires computations in the order of ˜O(2NₛNₚ). Finding the eigenvalues and eigenvectors, for example, using a cyclic Jacobi method [25], requires O(Nₛ³+N₃²N_{P}) computations. For projection, an O(2NₛN_{P}) operations are performed to reduce the dimensionality of spike waveforms to a 2-dimensional feature space.

A cluster-cutting algorithm, such as expectation-maximization (EM), is performed on the obtained 2-D feature space. Optimizing EM clustering requires ˜O(d²N_{P}²P) computations, where P here indicates the number of Gaussian models and d is the dimension of data (here d=2). To detect various spike prototypes, the EM clustering is implemented for different P's, and the best fit is selected. The overall computations required for EM clustering for a maximum number of P units is in the order of ˜Σ_{k=1 . . . P} O(4N_{P}²k)=O(2N_{P}²(P+1)P). Consequently, the overall computations required for training the PCA-based spike sorting is ˜O(4NₛN_{P}+Nₛ³+Nₛ²N_{P}+2N_{P}²(P+1)P). In the runtime mode, detected spikes are aligned and projected, and then classified to one of the predefined units using the Bayesian classifier, requiring computations in the order of ˜O(4Nₛ+4P).

In contrast, a five-level wavelet decomposition requires operations in the order of ˜O(23Nₛ) if classical convolution is used. However, this number can be significantly reduced by using the example embodiment Applicants reported in. Local averaging, typically used to remedy the shift variance property of the DWT, with a node-dependent filter requires computations in the order of ˜O(8Nₛ), since this filter is only applied to nodes 4, 6, 8, 9, and 10 in which spike features are mostly captured. At each node, one unit is discriminated at a time using a 2-class cluster cutting (binary classification). The required computations for this are in the order of ˜5×O(2N_{P}²). Consequently, the overall computations required for the training mode is in the order of ˜O(31NₛN_{P}+10N_{P}²). In the runtime mode, every detected event is decomposed, filtered, and classified using a 1-D Bayesian classifier with computations in the order of ˜O(31Nₛ+P).

For rate estimation, three methods were considered: the rectangular kernel (rate histogram), the Gaussian kernel and the extended. DWT (EDWT) Applicants propose. In EDWT, the firing rate is directly obtained by normalizing the threshold vectors and extending the decomposition to lower levels (higher frequency resolution). This requires ˜

$$O ( 45 {N}_{s} {n}_{w} {\sum }_{I = 5}^{∞} {2}^{- I} ) = O ( 22.5 \times {N}_{s} ) .$$

In the kernel based methods, a kernel function is convolved with the spike train and the rate is estimated by sampling the result. Assuming 45 ms bin width, and 2 ms refractory period, the number of computation required is in the order of ˜O(22.5×n_{w}). A Gaussian kernel width of n_{W}=100 is typically used to limit the amount of computations. The computational cost comparison is summarized in Table 1 and further plotted in the results section.

TABLE 1
COMPUTATIONAL COST FOR THE
TRAINING AND RUNTIME MODES

| | Training mode | Runtime mode |
|---|---|---|
| PCA/EM | O(4NₛN_{P} + Nₛ³ + | O(4Nₛ + 4P + 22.5n_{W}) |
| | Nₛ²N_{P} + 2N_{P}²(P + 1)P) | |
| Compressed sensing | O(21NₛN_{P} + 10N_{P}²) | O(43.5Nₛ + P) |

### II. Methods

Because our purpose was to demonstrate the ability to decode movement trajectory directly from neural data using the compressed signal representation, and given that the nature of cortical encoding of movement remains a subject of current debate in the neuroscience community, investigation of the methods developed in this paper required generation of neural data with known spike train encoding properties. This section describes in details the methods according to some example embodiments to model and analyze the data to demonstrate the validity of the approach.

### A. Spike Class Generation and Separability

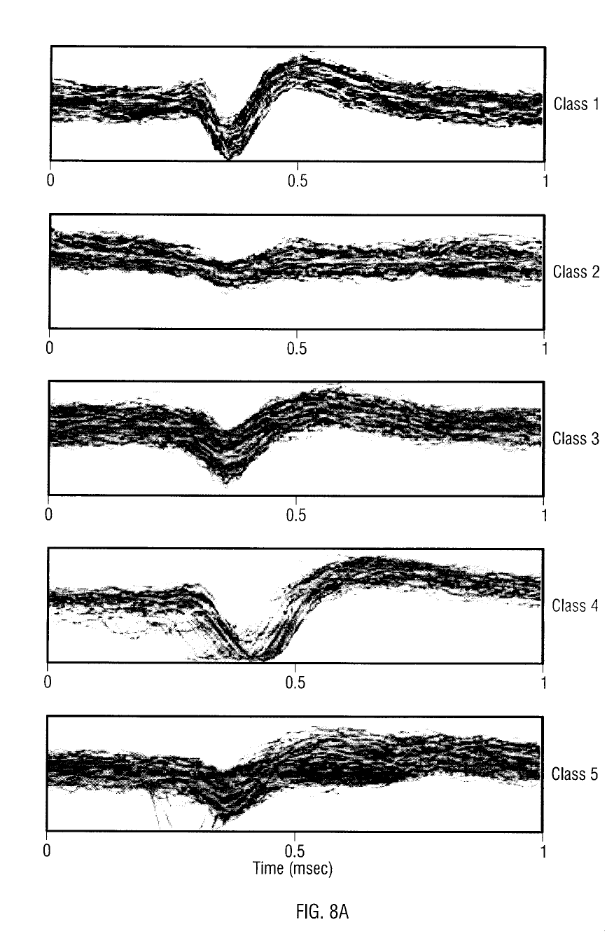

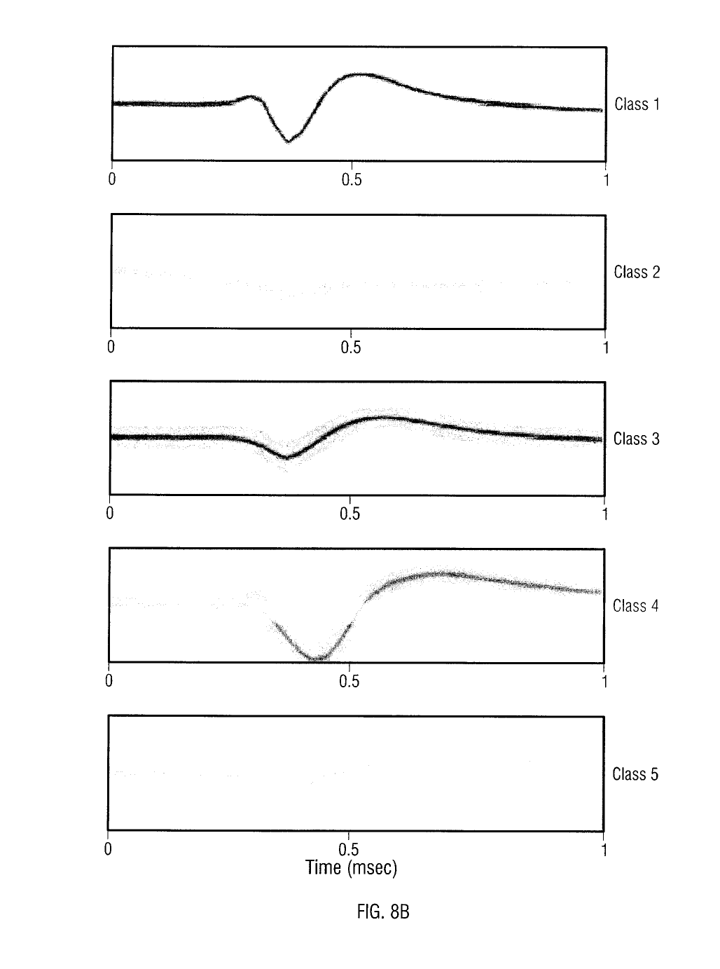

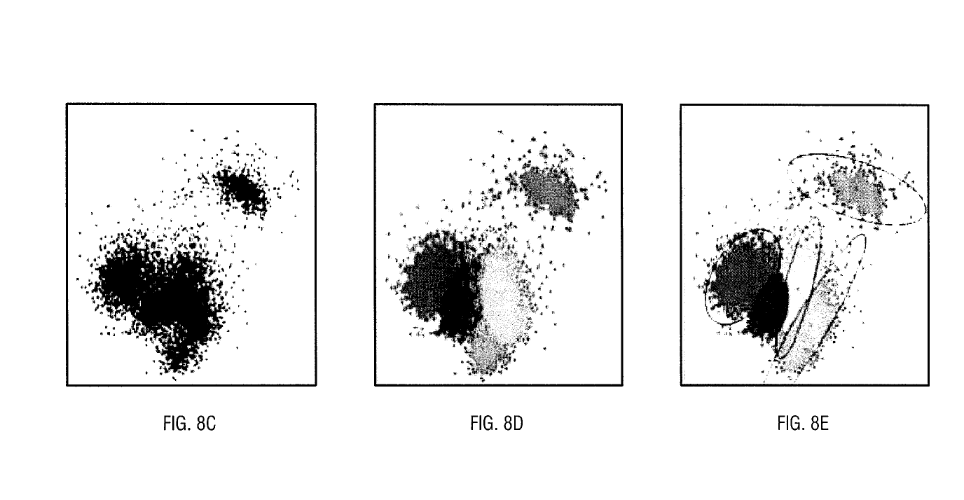

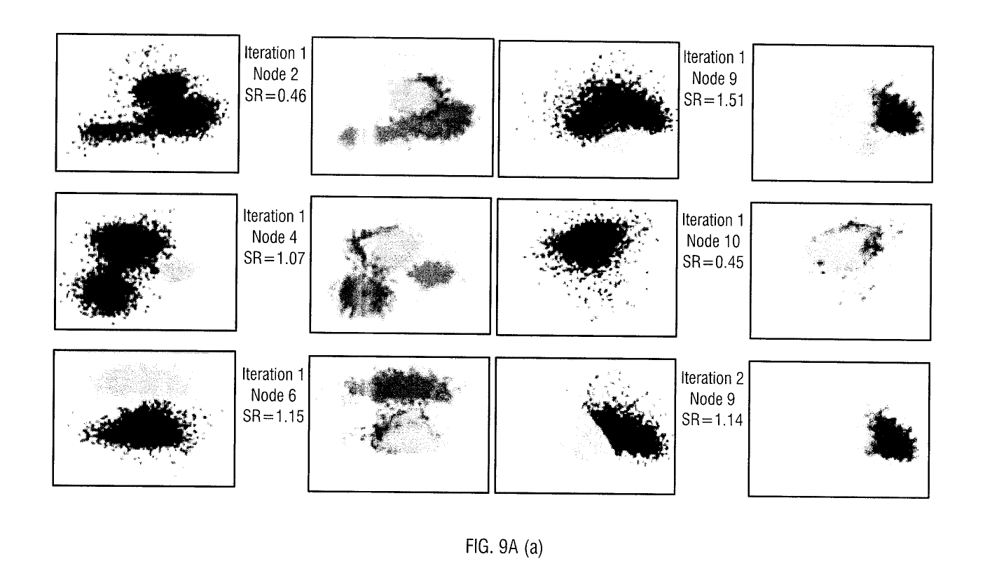

Spike waveforms were detected and extracted from spontaneous activity recorded in the primary motor cortex of an anesthetized rat using a 16-channel microelectrode array. All procedures were approved by the Institutional Animal Care and Use Committee at Michigan State University following NIH guidelines. Details of the experimental procedures to obtain these recordings are described elsewhere. These spikes were manually aligned and sorted using a custom spike sorting algorithm. Out of 24 units recorded, the actual action potential waveforms are shown in FIG. 8 for five representative units recorded on one electrode.

The separability of spike classes was calculated to determine the sensing thresholds for each neuron at any given time scale j. Specifically, in one example embodiment, the following measure

$$Γ \{ C \} = \frac{Between Cluster Separability}{Within Cluster Separability} = \frac{{S}_{B}}{{S}_{W}} \qquad ( 8 )$$

may be used for a set of clusters, {Cᵢ|i=1,2, . . . , P}. The between-cluster separability is defined as

$${S}_{B} = {\sum }_{i = 1}^{P} \frac{{\sum }_{x ∈ {C}_{i}} {\sum }_{y ∉ {C}_{i}} ‖ x - y ‖}{\vert {C}_{i} \vert {\sum }_{j \neq i} \vert {C}_{j} \vert} \qquad ( 9 )$$

where |Cᵢ| equals the number of spikes belonging to cluster Cᵢ, x and y are elements from the set of all spike waveforms and ∥·∥ represents the Euclidean distance (l₂ norm) between two elements. The quantity in (9) provides a factor proportional to the overall separation between clusters. For improved separability, a large S_{B} is desired. On the other hand, the within-cluster separability is defined as

$${S}_{W} = {\sum }_{i = 1}^{P} \frac{{\sum }_{x ∈ {C}_{i}} {\sum }_{y ∈ {C}_{i}} ‖ x - y ‖}{\vert {C}_{i} \vert ( \vert {C}_{i} \vert - 1 )} \qquad ( 10 )$$

and is proportional to the overall spread within each of the individual clusters. For improved separability, a small S_{W} is desired. Therefore, a large Γ indicates a greater overall separability.

In one example embodiment, a separability ratio (SR) may be computed as the ratio between Γ{2} (i.e. a 2-class separability) in every node to that in the time domain. Therefore, an SR ratio of 1 indicates equal degree of separability in both domains, while ratios larger than 1 indicate superior separability in the sparse representation domain. This later case implies that at least one unit may be separated in that node's feature space better than the time domain's feature space. This detected unit is subsequently removed from the data and the decomposition process continues until all possible units are detected, or all nodes have been examined on any given electrode. On the other hand, if the same unit can be discriminated in more than one node, the “best node” for discrimination of this unit is the node that provides the largest SR. For a given probability of False Positives (typically 0.1), the sensing threshold γₚ^{j} is determined by maximizing the separability of at least one spike class in each node. Since the sensing threshold is chosen to discriminate between spike events and not to minimize the MSE of the reconstructed spike, this selection rule results in thresholds that are typically higher than those obtained from the universal thresholding rule for denoising and near-optimal signal reconstruction. As a result, the number of false positives that may be caused by classifying noise patterns as unit-generated spikes is automatically reduced.

### B. Population Model of 2D Arm Movement

In one example embodiment, to simulate spike trains from motor cortex neurons during movement planning and execution, a probabilistic population encoding model of a natural, non-goal directed, 2D arm movement trajectory may be used. The arm movement data were experimentally collected to ensure realistic kinematics. The discrete time representation of the conditional intensity governing each neuron firing rate was modeled as a variant of the cosine tuning model of the neuron's preferred direction θₚ (ranging from 0 to 2π).

$${λ}_{p} ( {t}_{k} \vert {x}_{p} ) = exp ( {β}_{p} + {δ}_{p} \dot{θ} cos ( \frac{θ ( {t}_{k} ) - {θ}_{p}}{{ω}_{p}} ) ) p = 1 , 2 , … , P \qquad ( 11 )$$

where βₚ denotes the background firing rate, θ(tₖ) denotes the actual movement direction, θ denotes velocity magnitude (kept constant during the simulation), xₚ=[θₚ, δₚ, ωₚ] is a parameter vector governing the tuning characteristics of neuron p, where it was assumed that the tuning depth δₚ was constant (δₚ and βₚ where fixed for all neurons and equal to 1 and log(5), respectively), the preferred direction θₚ was uniformly distributed, while the tuning width ωₚ was varied across experiments. Using this model, event times were obtained using an inhomogeneous Poisson process with 2 ms refractory period as

Pr{spike from neuron p in (tₖ,tₖ+Δ]}≈λₚ(tₖ)·Δ (12)

where Δ is a very small bin (−1 ms).

The tuning term in (11) incorporates a neuron-dependent tuning width ωₚ, an important parameter that affects the bin width choice for rate estimation prior to decoding. Variability in this term (ωₚ ranged from 0.25 to 4 in each experiment) resulted in firing rates that are more stochastic in nature and served to closely approximate the characteristics of cortical neurons' firing patterns. In some example embodiment, the mean squared error between the rate functions obtained from the simulated trajectory data and the estimated rates may be defined as:

$${MSE}_{j} = \frac{1}{N} {\sum }_{n = 1}^{N} {( λ [ n ] - {\hat{λ}}_{j} [ n ] )}^{2} j = 1 , 2 , … , J \qquad ( 13 )$$

While equation (13) provides a simple and obvious measure of performance, in practice the true rate function may be unknown. Information theoretic measures are useful in such cases since they assess higher order statistical correlation between the estimators and measurable quantities such as the observed movement and can be useful to determine the time scale that best characterize the information in the instantaneous firing rate. In some example embodiment, a node-dependent mutual information metric between the encoded movement parameter and the rate estimator may be defined as:

$${I}_{j} = {\sum }_{θ , {\hat{λ}}_{j}} p ( θ , {\hat{λ}}_{j} ) log \frac{p ( θ , {\hat{λ}}_{j} )}{p ( θ ) p ( {\hat{λ}}_{j} )} , j = 1 , 2 , … , J \qquad ( 14 )$$

This metric is particularly useful when the instantaneous rate function is not Gaussian distributed.

### III. Results

### A. Spike Class Separability

FIG. 8(c) shows a scatter plot of the first two principal components of the five representative spike classes in FIG. 8(a). Consider for example unit 4 that appears quite well isolated in the time domain feature space, It is clear that the other classes are poorly isolated. Results of manual, extensive, offline sorting using hierarchical clustering of all the features in the data are displayed in FIG. 8(d). In FIG. 8(e), the clustering result using automated, online PCA/EM cluster-cutting with two principal features is illustrated. Examination of these FIGS. reveals that the lack of separability in the feature space, particularly for units 1, 2, 3 and 5, results in significant differences between the manual, extensive, offline sorting result and the automated online PCA/EM result.

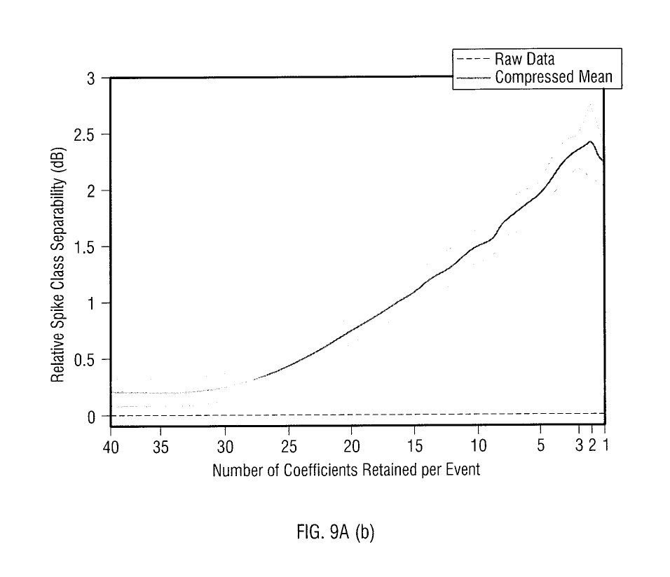

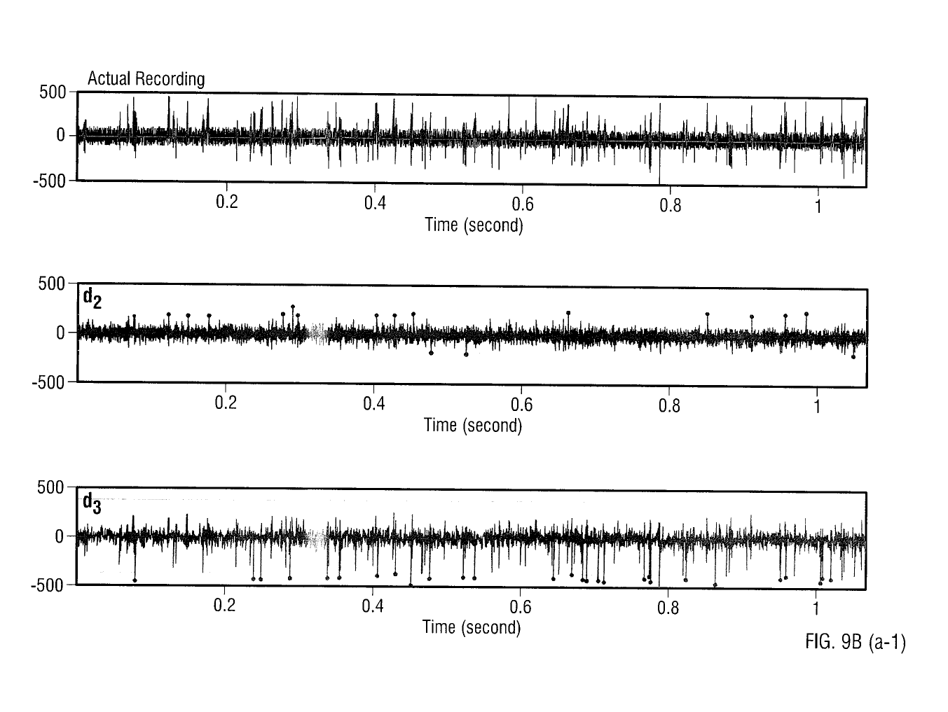

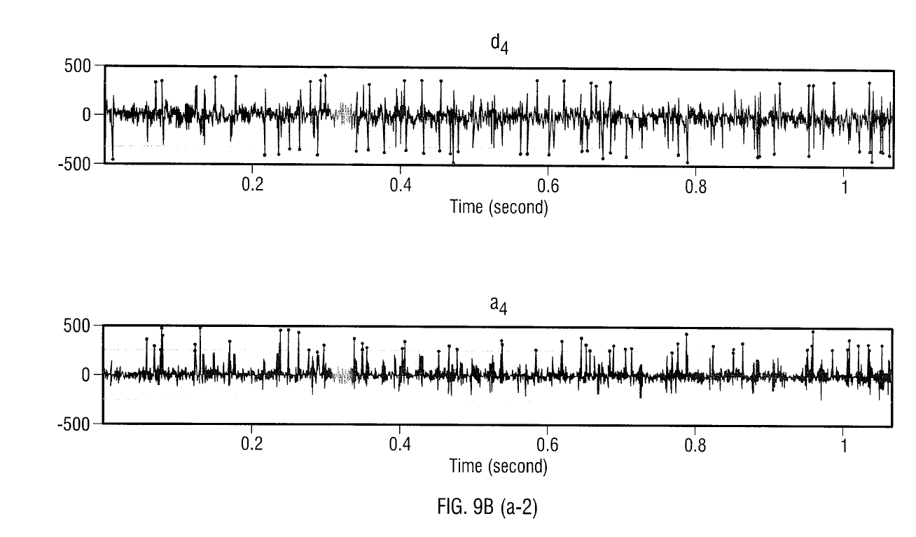

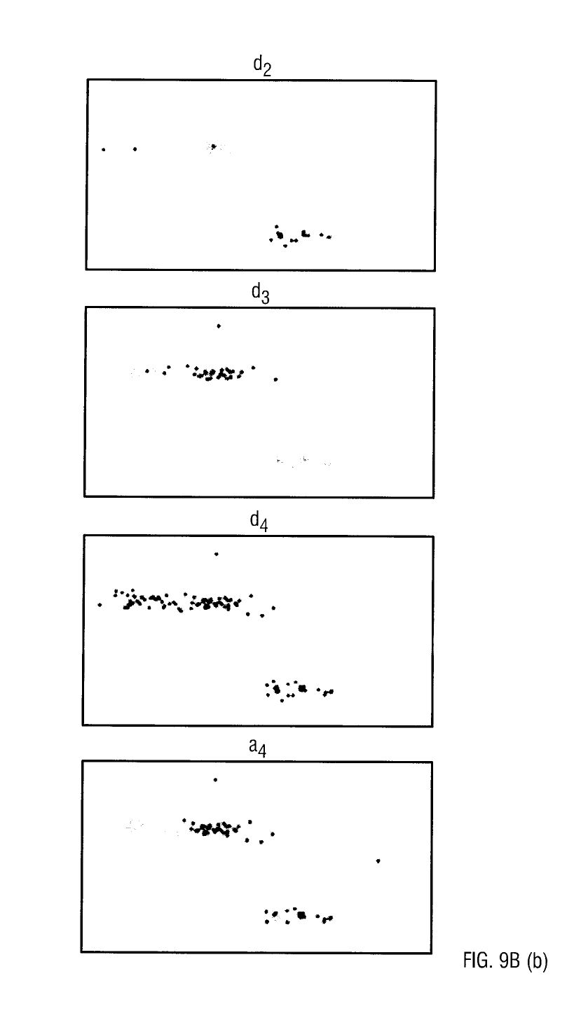

Alternatively, when a two-class situation is considered where one single cluster is isolated in a given node while all other spike classes are lumped together, FIG. 9A illustrates that each spike class is separable in at least one node of the sparse representation. The different degrees of separability across nodes permit isolating one class at a time, owing to the compactness property of the transform in nodes that are best representative of each class. For example, class 1 appears poorly isolated from class 5 in the time-domain feature space, yet it is well separated from all the other classes in node 6.

It can be seen from (a) of FIG. 9A that in most nodes, the SR ratio is larger than 1 (except for nodes 2 and 10). For the 24 units recorded in this data set, the performance of the compressed sensing strategy was 92.88+6.33% compared to 93.49+6.36% for the PCA-EM. Performance of the sensing threshold selection process was quantified as a function of the number of coefficients retained in (b) of FIG. 9A. As the sensing threshold is increased, the number of retained coefficients logically decreases thereby improving compression. However, the most interesting result is the improved separability by more than 70% compared to time domain separability at roughly 97% compression. This implies that discarding some of the coefficients that may be needed for optimal spike reconstruction and sorting in the time domain in a classical sense does improve the ability to discriminate between spike classes based on their magnitude only. Maximum separability is reached when a few coefficients/event is retained, after which some classes are entirely lost and the performance deteriorates.

### B. Firing Rate Estimation

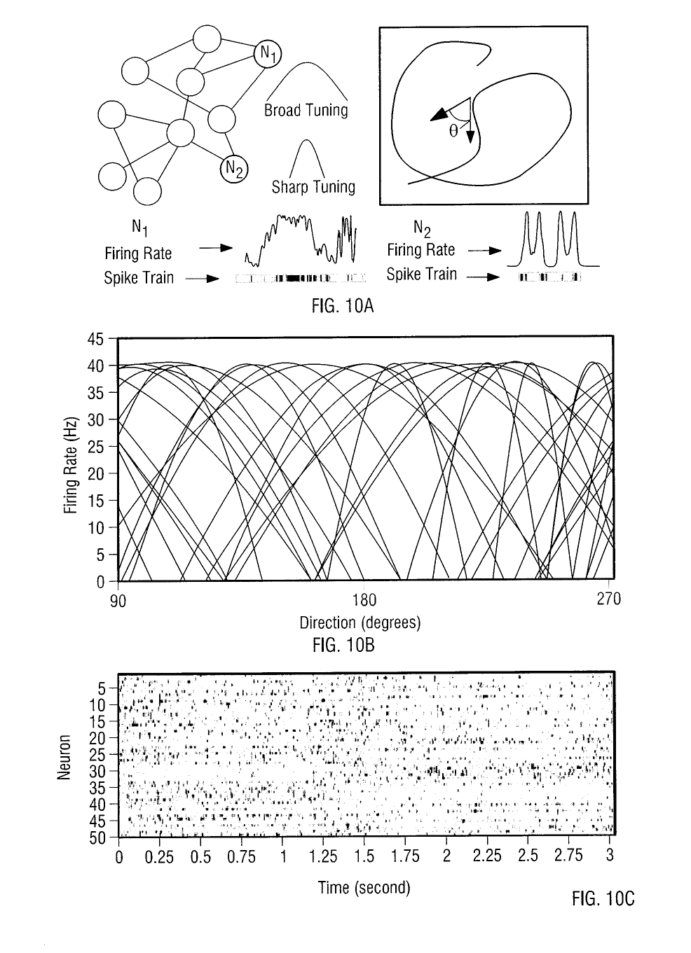

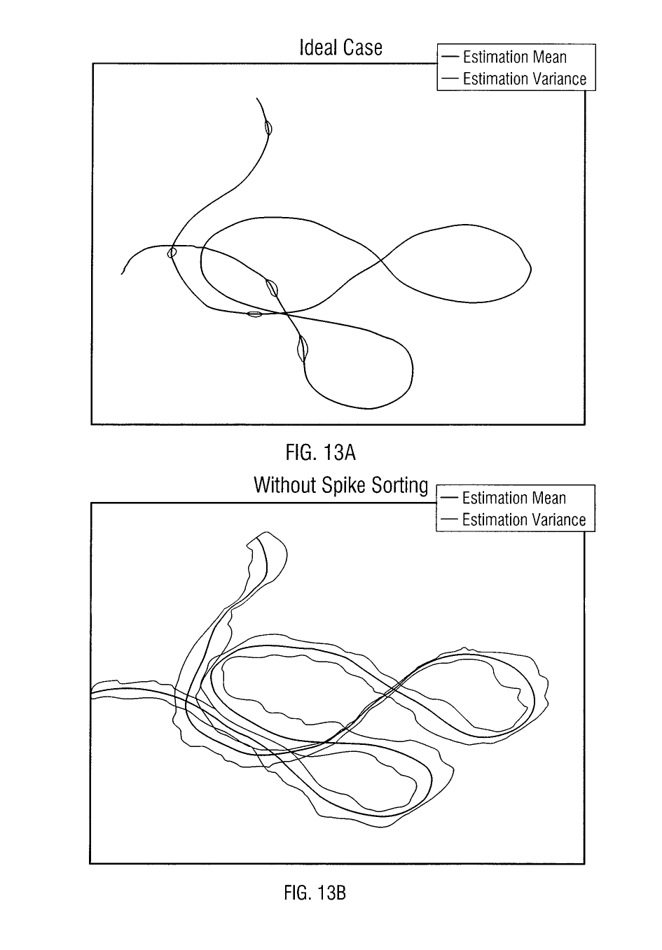

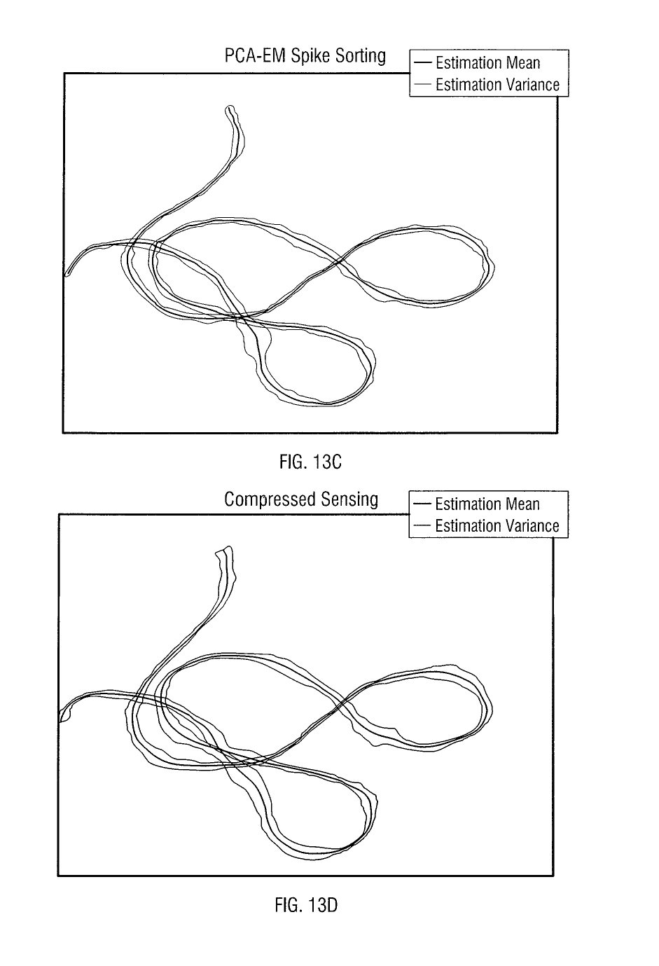

A sample trajectory, rate functions from neurons with distinct tuning characteristics and their spike train realizations are shown in FIG. 10. It can be clearly seen in FIG. 10(a) that the tuning width has a direct influence on the spike train statistics, particularly the ISI. A broadly tuned neuron exhibits more regular ISI distribution, while a sharply tuned neuron exhibits a more irregular pattern of ISI. FIG. 10(b) illustrates the tuning characteristics of a subpopulation of the entire population over a limited range (for clarity) to demonstrate the heterogeneous characteristics of the model Applicants employed. A 9-second raster plot in FIG. 10(c) illustrates the stochastic patterns obtained for the trajectory illustrated later in FIG. 13.

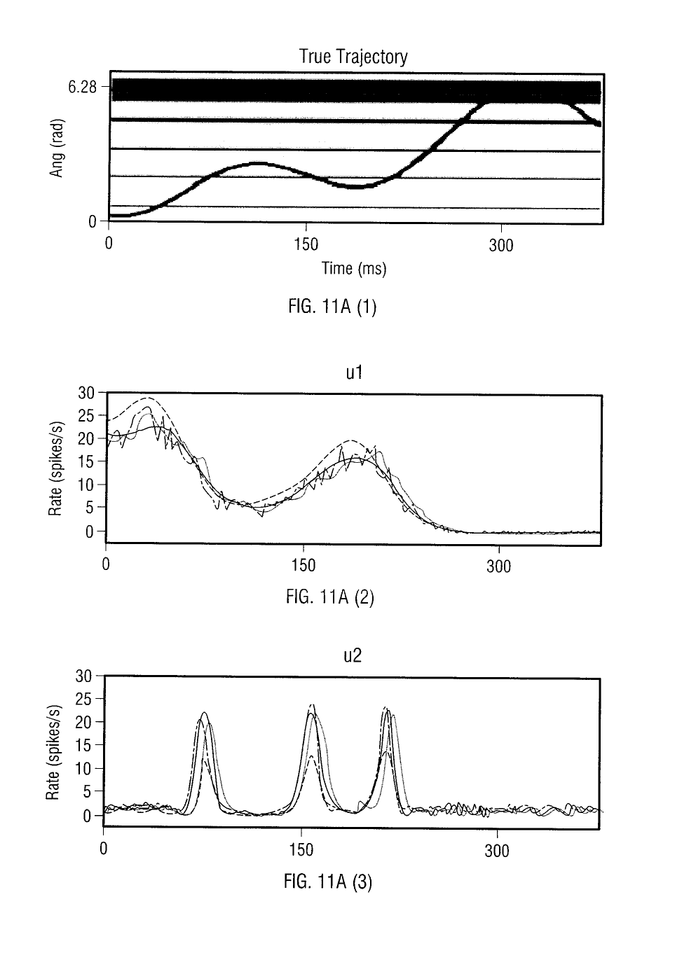

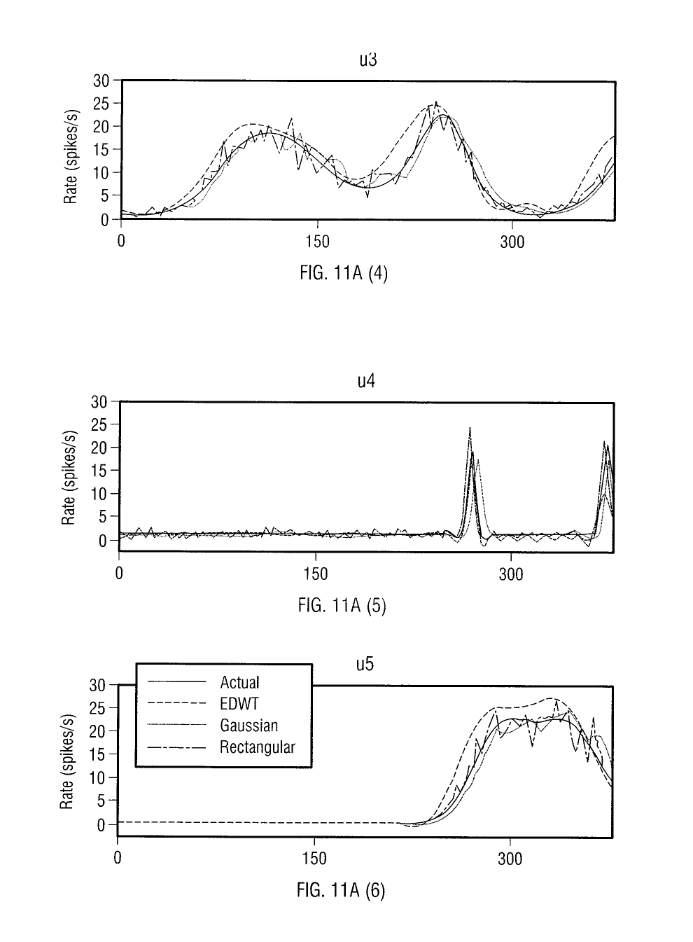

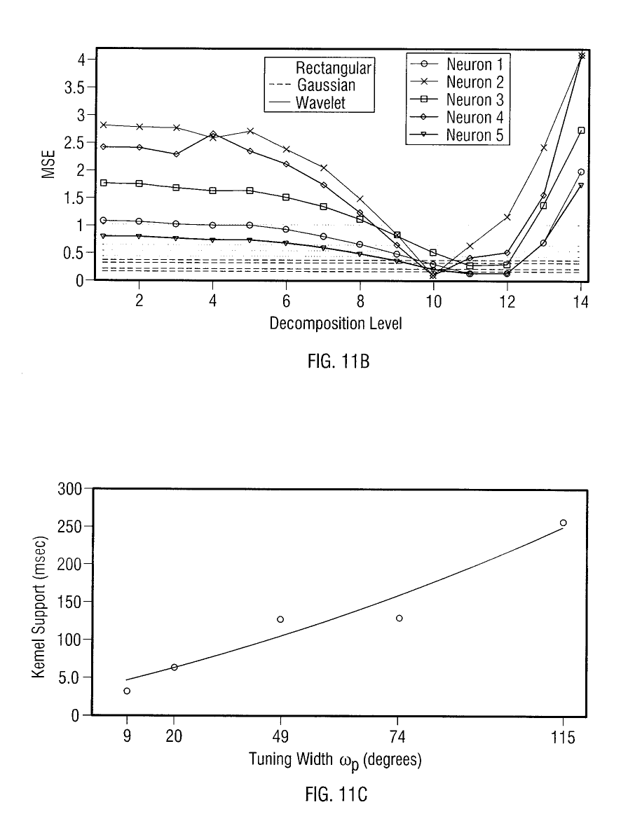

In FIG. 11, a 300-msec segment of the movement's angular direction over time is illustrated superimposed on the neuronal tuning range of five representative units with distinct tuning widths. The resulting firing rates and their estimators using the rate histogram, Gaussian kernel, and extended DWT methods are illustrated for the five units, showing various degrees of estimation quality. As expected, the rate histogram estimate is noisy, while the Gaussian and EDWT methods perform better. In FIG. 11(b), the relation between the wavelet kernel size and the MSE is quantified. As expected, decomposition levels with shorter kernel width (i.e., fine time scales) tend to provide the lowest MSE for neurons that are sharply tuned.

In contrast, a global minimum in the MSE is observed for broadly tuned neurons at coarser time scales, suggesting that these decomposition levels may be better suited for capturing the time varying-characteristics of the firing rates. Interestingly, the MSE for the EDWT method attains a lower level than both the rectangular and Gaussian kernel methods at the optimal time scale, clearly demonstrating the superiority of the proposed approach. The relation between the tuning width and the kernel size for the entire population is illustrated in FIG. 11(c). As the tuning broadens, larger kernel sizes (i.e. deeper decomposition levels) are required to attain a minimum MSE and thus better performance.

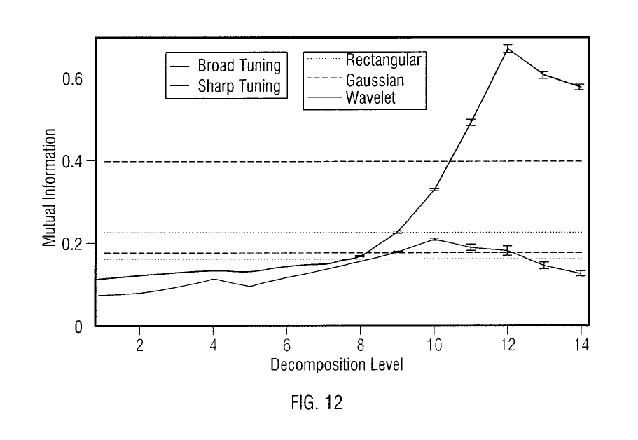

The mutual information between the actual movement trajectory and the rate estimators are shown in FIG. 12. There is a steady increase in the mutual information versus kernel support until a maximum is reached at the optimal decomposition level that agrees with the minimum MSE performance. This maximum coincides with a rate estimator spectral bandwidth matching that of the underlying movement parameter. Rate estimators beyond the optimal time scale do not carry any additional information about the movement trajectory.

### C. Decoding Performance

A sample trajectory and the decoded trajectory are shown in FIG. 13 for four different cases: First, when no spike sorting is required. This is the ideal case in which every electrode records exactly the activity of one unit, but is hard to encounter in practice. Second, when two or more units are recorded on a single electrode but no spike sorting is performed prior to rate estimation. Third, when spike sorting is performed for the latter case using the PCA/EM/Gaussian kernel algorithm. And fourth, when combined spike sorting and rate estimation are performed using the compressed sensing method. Applicants used a linear filter for decoding in all cases [30]. It is clear that the proposed method has a decoding error variance that is comparable to the PCA/EM/Gaussian kernel algorithm, suggesting that the performance is as good as, if not superior, to the standard method.

### D. Computational Cost

An important aspect to validate and confirm the superiority of our approach is to compare the computational complexity of the standard PCA/EM/Gaussian kernel rate estimator to the compressed sensing method for different event lengths (Nₛ) and different number of events (Nₚ) per neuron.

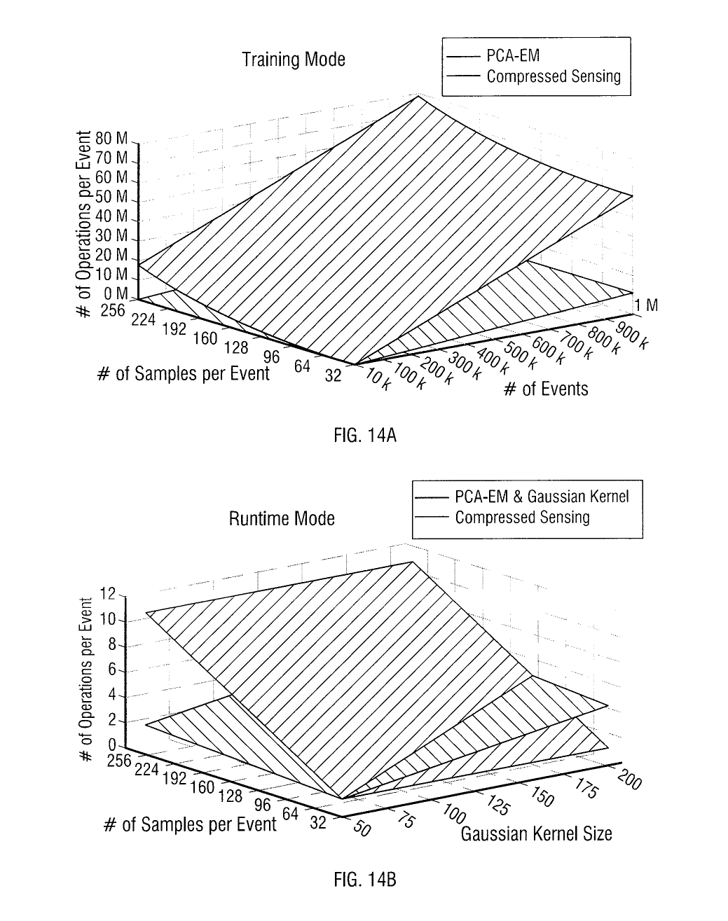

The results illustrated in FIG. 14 show that the proposed method requires significantly less computations for training. This is mainly attributed to the complexity in computing the eigenvectors of the spike data every time a new unit is recorded. In contrast, wavelets are universal approximators to a wide variety of transient signals and therefore do not need to be updated with the occurrence of events from new units. In the runtime mode, the computational cost for the proposed method becomes higher when the number of samples/event exceeds 128 samples. At a nominal sampling rate of 40 kHz (lower rates are typically used), this corresponds to a 3.2 ms interval, which is much larger than the typical action potential duration (estimated to be between 1.2-1.5 msec).

### IV. Discussion

Applicants have proposed a new approach to directly estimate a critical neuronal response property—the instantaneous firing rate—from a compressed representation of the recorded neural data. The approach has three major benefits: First, the near-optimal denoising and compression allows to efficiently transmit the activity of large populations of neurons while simultaneously maintain features of their individual spike waveforms necessary for spike sorting, if desired. Second, firing rates are estimated across a multitude of timescales, an essential feature to cope with the heterogeneous tuning characteristics of motor cortex neurons. These characteristics are important to consider in long term experiments where plasticity in the ensemble interaction is likely to affect the optimal time scale for rate estimation. Third, as our extensive body of prior work has demonstrated [11, 31], the algorithm can be efficiently implemented in low-power, small size electronics to enable direct decoding of the neural signals to take place without the need for massive computing power. Taken together, these are highly desirable features for real-time adaptive decoding in BMI applications.

Applicants have used a particular model for encoding the 2D hand trajectory for demonstration purposes only. It should be noted, however, that the method is completely independent of that model. In one example embodiment, the sparse representation may preserve all the information that needs to be extracted from the recorded neural data to permit faithful decoding to take place downstream. This includes the features of the spike waveforms as well as the temporal characteristics of the underlying rate functions.

In the tests performed here Applicants have used the same wavelet basis—the symmlet4—for both spike sorting and rate estimation. This basis was previously demonstrated to be near-optimal for denoising, compression, and hardware implementation. However, the possibility exists to use this basis in the first few levels, and then extend the decomposition from that point on using a different basis that may better represent other features present in the rate functions that were not best approximated by the symmlet4. For example, the “bumps” in the sparse rate estimates in FIG. 11 are not as symmetrical in shape as those in the original rate, or those in the Gaussian estimator. For this particular example a more symmetric basis may be better suited.

Estimation of the rate using a fixed bin width may be adequate for certain applications that utilize firing rates as the sole information for decoding cortical responses during instructed behavioral tasks such as goal-directed arm reach tasks [2-4, 32]. These operate over a limited range of behavioral time scales. However, natural motor behavior is characterized by more heterogeneous temporal characteristics that reflect highly-nonstationary sensory feedback mechanisms from the surrounding cortical areas. The firing rates of motor neurons during naturalistic movements are highly stochastic and require a statistically-driven technique that can adapt to the expected variability [18, 33]. This is particularly important given the significant degrees of synchrony typically observed between cortical neurons during movement preparation [34], and also observed during expected and unexpected transitions between behavioral goal representations [35].

While it has been argued that precise spike timing does not carry information about motor encoding [36], one must note that most of the BMI demonstrations to date were carried out in highly-trained subjects performing highly stereotypical, goal-directed behavioral tasks. Very few studies, if any, have been carried out to characterize naturally occurring movements in naïve subjects. Thus, the potential still exists for new studies that may demonstrate the utility of both neuronal response properties, namely precise spike timing and firing rate, in decoding cortical activity. For that, the sparse representation is able to simultaneously extract these two important elements that are widely believed to be the core of the neural code [37]. Therefore, our proposed approach is the first to offer the solution for extracting both properties within a single computational platform in future generations of BMI systems.

It is noted that for a fully implantable interface to the cortex to be clinically viable, spike detection, sorting, and instantaneous rate estimation need to be implemented within miniaturized electronics that dissipate very low power in the surrounding brain tissue. More recently, it has been shown that tethering the device to the subject's skull to maintain a wired connection to the implant significantly increases brain tissue adverse reaction, which is believed to negatively affect implant longevity [38]. Therefore, the interface needs to feature wireless telemetry to minimize any potential risk of infection and discomfort to the patient and to elongate the implant's lifespan. It is noted that eliminating any of the steps from the signal processing path while preserving the critical information in the neural data will significantly reduce the computational overhead to permit small size, low power electronics to be deployed and accelerate the translation of this promising technology to clinical use.

### V. Conclusion

Applicants have proposed a new approach to directly estimate instantaneous firing rates of cortical neurons from their compressed extracellular spike recordings. The approach is based on a sparse representation of the data and eliminates multiple blocks from the signal processing path in BMI systems. In some example embodiment, Applicants used the decoding of simulated 2D arm trajectories to demonstrate the quality of decoding obtained using this approach. Applicants also demonstrated that regardless of the type of neural response property estimated, the approach efficiently captures the intrinsic elements of these responses in a simple, adaptive, and computationally efficient manner. The approach was compared to other methods classically used to estimate firing rates through a more complex processing path. Applicants further demonstrated the improved performance attained with Applicants' approach according to some example embodiments, while maintaining a much lower computational complexity.

Quantitative measures were applied to show that the sparse representation allows for better unit separation compared to classical PCA techniques, currently employed by many commercial data acquisition systems. This suggests that full reconstruction of the spike waveforms for traditional time domain sorting is not necessary, and that more accurate spike sorting performance could ultimately be achieved when the proposed method is used. This translates into substantial savings in computational and communication costs for implantable neural prosthetic systems to further improve their performance and potential use in clinical applications.

### VI. Spike Sorting Algorithm Hardware Configurations

Tradeoffs between computational complexity and stringent design constraints of an implantable system are unavoidable. As discussed above, new algorithms provide at least one solution, wherein a large compression of neural data can be achieved prior to telemetry transmission. Oweiss K., “A systems approach for data compression and latency reduction in cortically controlled brain machine interfaces,” IEEE Transactions on Biomedical Engineering 531364-1377 (2006). Further, compromises among power, size and speed of computation can be achieved within an optimized hardware implementation. Oweiss et al., “A scalable wavelet transform VLSI architecture for real-time signal processing in high-density intra-cortical implants” IEEE Transactions on Circuits and Systems 54:1266 (2007). For example, sparse representation analysis not only overcomes severe bandwidth limitations of a wireless implantable system, but also provides efficient spike sorting without the need to decompress and reconstruct spike waveforms. Aghagolzadeh et al., “Compressed and Distributed Sensing of Neuronal Activity for Real Time Spike Train Decoding” IEEE Transactions on Neural Systems and Rehabilitation Engineering 17:116-127 (2009).

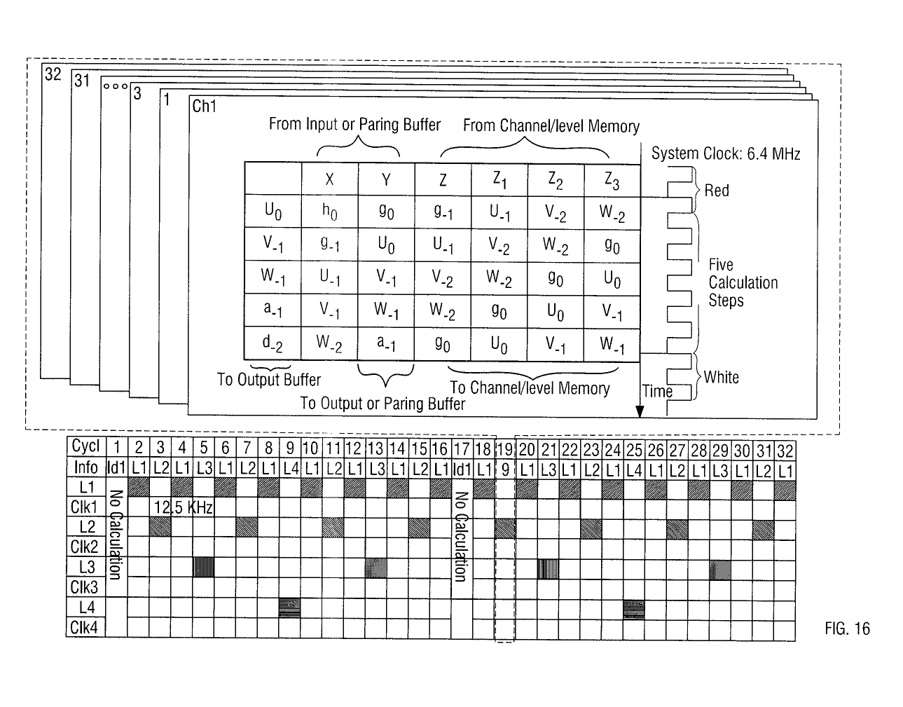

In one example embodiment, t an implantable device comprising a hardware architecture configured to support efficient spike sorting using sparse representation analysis is provided. In one embodiment, the sparse representation analysis comprises a compressive spike sorting algorithm module. See, for example, FIG. 16.

A. One-Dimensional Spike Sorting

To be hardware friendly, spike sorting needs to be implemented based on a small set of features—eventually a single feature per waveform. In such case, this feature would be compared to a threshold, which can be implemented using a very simple comparator circuit. Sparse representation analysis using discrete wavelet transform (DWT) can obtain this single feature for each spike waveform, because it carries information about spike times at fine resolutions, while carrying information about spike shape at coarser resolutions. Mathematically, a DWT decomposition of a spike waveform, xₜ, is expressed as

$${x}_{t} = {\sum }_{t - 1}^{L} ( {\sum }_{k} {a}_{tj λ} {ψ}_{jk} + {\sum }_{k} {d}_{tj , k} {ψ}_{jk} )$$

where L determines the number of decomposition levels (i.e., for example, ranging between one to five levels), aₜⱼ=(xₜ, φⱼ). and dₜⱼ=(xₜ, ψⱼ) are the approximations and detail coefficients, respectively; {.,.} denotes the dot product, and φ and ψ are the low-pass and high-pass filters obtained from the symlet4 wavelet basis. Mallat, S., “A wavelet tour of signal processing” Academic Press (1999). The detail coefficients of levels 2, 3 and 4, and the approximation coefficients of level 4, referred to as nodes 4, 6, 8 and 7, respectively, are used for sorting the waveforms. The magnitude of the largest DWT coefficient in each node is selected as the single feature to be compared to a predetermined threshold. Selecting a single feature per waveform in each DWT level allows us to express the sorting problem as a node-dependent binary hypothesis testing problem

Selecting a single feature per waveform in each DWT level allows us to express the sorting problem as a node-dependent binary hypothesis testing problem:

H₁:x=sₜ+n

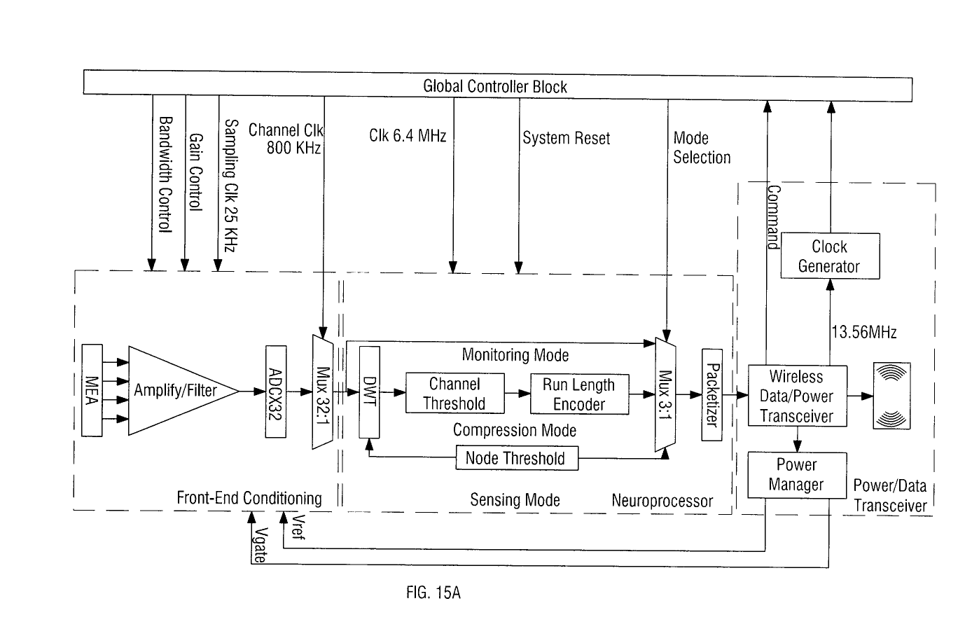

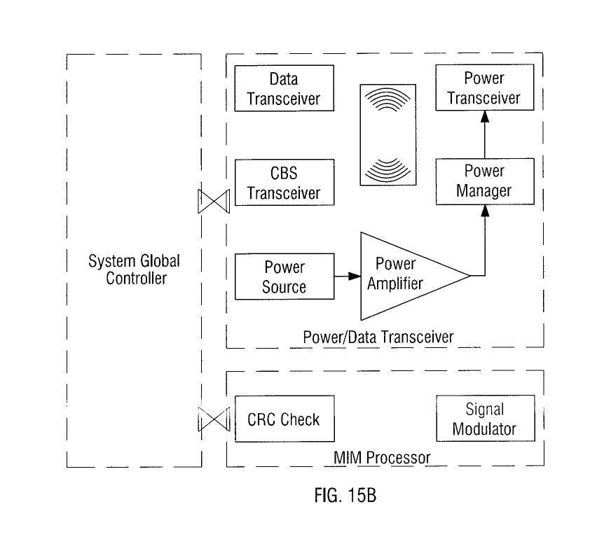

H₀:x={sⱼ}_{j≠1}+n

where xεX is the output of the DWT block. See, FIG. 15A. sᵢ is the single feature extracted from neuron i's spike waveform, {sⱼ}_{j≠} indicates similar features extracted from other neurons except neuron i, and n expresses a noise term. A decision rule based on a Likelihood-ratio test (LRT), Λ(x), is expressed as:

$$Λ ( x ) = \frac{{P}_{1} ( {s}_{i} \vert x )}{{P}_{0} ( {s}_{j} \vert x )} {≷}_{{H}_{0}}^{{H}_{1}} Y i$$

Van Trees H., “Detection, estimation, and modulation theory” Wiley-Interscience (2001) where γₗ, is a node-specific threshold for node l, and Pₖ(sᵢ|x) is the posterior density of sᵢ given x, under Hₖ. Using Bayes theorem, the posterior is a function of the likelihood, Pₖ(sᵢ|x), as:

$$P ( {s}_{i} \vert x ) = \frac{P ( {s}_{i} ) P ( x \vert {s}_{i} )}{{\sum }_{{s}_{i} ∈ S} P ( {s}_{i} ) P ( x \vert {s}_{i} )}$$

where P(sⱼ) is the probability of firing for neuron i. Therefore, in the presence of N neurons, N two-class classifiers are needed, where each binary classifier operates in one node of the DWT and separates one spike train per node. Aghagolzadeh et al., “Compressed and Distributed Sensing of Neuronal Activity for Real Time Spike Train Decoding,” IEEE Transactions on Neural Systems and Rehabilitation Engineering 17:116-127 (2009).

B. Hardware Implementation

Briefly, a DWT module performs the DWT transformation simultaneously on 32 channels for up to 5 levels using the computationally efficient lifting method. Oweiss et al., “A scalable wavelet transform VLSI architecture for real-time signal processing in high-density intra-cortical implants” IEEE Transactions on Circuits and Systems 541266 (2007). In one example embodiment, a sequence of machine cycles for these five levels is provided (FIG. 16). In this sequence, an L1 coefficient is computed, once two samples are received, followed by an L2 coefficient for two computed L1 coefficients, and so on. The 32 machine cycles start with an idle (no calculation) cycle, marked as Idl. At a sampling rate of 25 kHz per channel, the entire system is clocked at a maximum 6.4 MHz frequency to ensure eight operation cycles required by the lifting method (2 cycles for reading, 5 cycles for computing and 1 cycle for writing the data).

To control the sequence and timing of operations within a compressive sorting module, a controller based on finite state machines is used. In this controller, an 8-bit counter is used to keep track of the channel and level information sequentially for example, 5 bits for a channel index, and 3 bits for a node index). Another 18-bit counter is used to keep track of the universal timing in the module. At 25 kHz sampling rate, this counter resets approximately every 10 seconds. This module is designed and simulated in Verilog with ModelSim XE III 6.4b. The implementation is synthesized and verified using the Altera Cyclone III FPGA evaluation board.

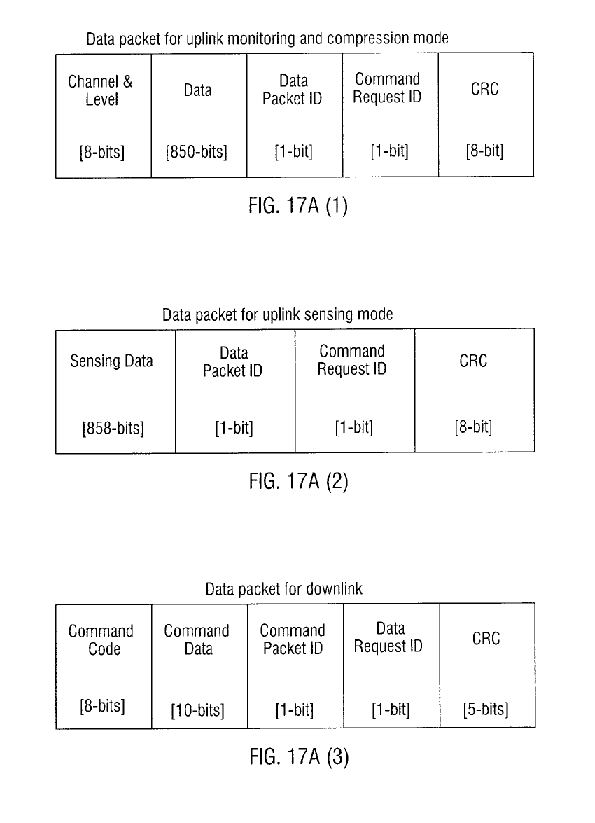

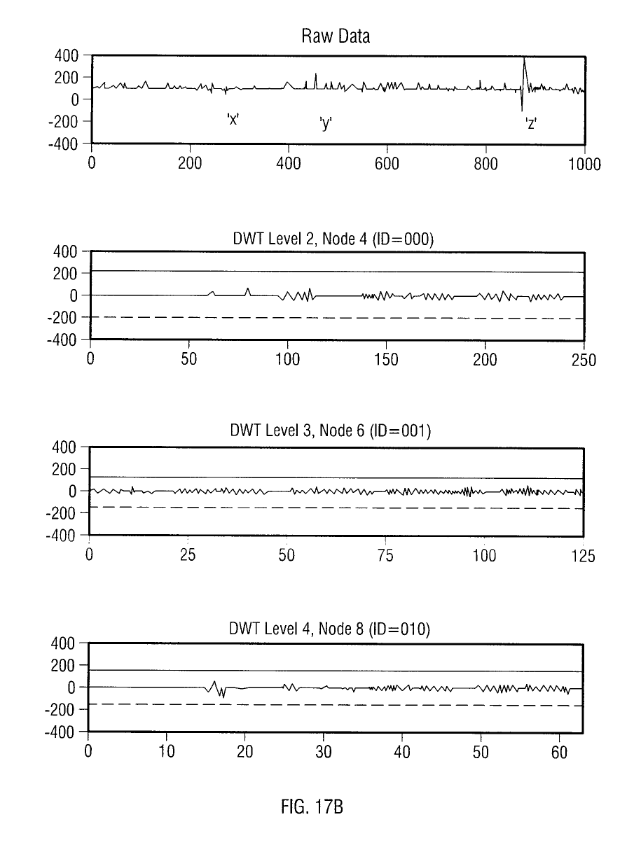

In one example embodiment, a compressive sorting module comprising a plurality of algorithm blocks is provided. In one embodiment, at least one block comprises a DWT for computing a plurality of wavelet coefficient. In one embodiment, at least one block comprises a comparator for detecting large coefficients. In one embodiment, at least one block comprises a RAM for storing a plurality of 32×5=160 node-specific thresholds (i.e., for example, for providing comparator input). In one embodiment, at least one block comprises a counter for tracking decomposition levels for each channel. See, FIG. 17A.

The entire module may operate in at least two modes; i) a programming mode, where thresholds are uploaded after the training period; and ii) a run-time mode, where estimated coefficients are compared with the stored thresholds. When the system is initially turned on, the programmable chip contains no information. The user sends a command to the chip to start the training period, during which the chip transmits enough data to an external computing device to train the binary classifiers and compute the optimal thresholds. Aghagolzadeh et al., “Compressed and Distributed Sensing of Neuronal Activity for Real Time Spike Train Decoding” IEEE Transactions on Neural Systems and Rehabilitation Engineering 17:116-127 (2009). These thresholds are then downloaded by a chip controller and stored into a RAM block (i.e., for example, programming mode). Then, coefficients corresponding to a particular channel and node are compared with these thresholds, so that large coefficients at the output of a comparator contain the information of spike events.

The detected events are then formatted individually into packets. See, Table. 2.

TABLE 2
Data Sample Format at the Output of Compressive Sorting Module

| Channel Index | Node Index | Time Index |
|---|---|---|
| [5-bits] | [3-bits] | [18-bits] |

In one embodiment, the length of each packet is 26 bits per even. In one embodiment, 5 bits are used to store the event's channel index. In one embodiment, 3 bits are used for an event's node index. In one embodiment, 18 bits are used for a time index. In one embodiment, an 18-bit universal counter is used to track an internal time index. Although not wishing to be bound by this proposed theory, it is believed that once the counter is full, it automatically resets and restarts counting, and keeping track of the exact timing is done externally using the transmitted time index. In one example embodiment, the spike trains are reconstructed as binary sequences, wherein the length of the universal counter is long enough to minimize the possibility of losing track of the exact timing by the observer.

C. In Vivo Data Recording

In one example embodiment, a method comprising recording neural spike waveforms from a mamma is provided. In one embodiment, the spikes are recorded from a mammalian brain. In one embodiment, the mammalian brain is a rat brain. In one embodiment, the mammalian brain is a human brain. In one embodiment, the recording is performed with a 32 channel microelectrode array. In one embodiment, the spikes are manually aligned. In one embodiment, the aligned spikes are sorted using a semi-automatic spike sorting algorithm.

A sample trace with three spike events from three distinct neurons were recorded on an emulated chip. A first row on the chip comprises a recorded spike train, wherein a plurality of individual events are labeled as ‘x’, ‘y’ and ‘z’. See, FIG. 17B. The detail coefficients of the spike train estimated by the DWT block are displayed for nodes 4, 6 and 8. The threshold imposed by the comparator is illustrated as the dashed lines for each node. At node 4, all three spike events are detected as the absolute magnitude of their coefficient surpassed the threshold. At node 6, however, only ‘x’ and ‘y’ surpassed the threshold, while only ‘y’ surpassed in node 8. Therefore, a total of 6 spike events were sent to a wireless transceiver module and transmitted to an external observer. At the destination, spike event ‘y’ is exclusively detected when a single DWT coefficient surpasses the node-specific threshold of node 8. Detected events around the same timestamp in the remaining nodes are discarded to prevent multiple counting of the same event. By eliminating the information about ‘y’, ‘x’ can be exclusively detected when a single DWT coefficient surpasses the node specific threshold of node 6. Similarly, event ‘z’ is detected at node 4, and so on.

To investigate the optimal bit precision that maintains the same classification performance as the offline system, Receiver Operating Characteristics (ROCs) were computed for different bit precisions of the data. The True Positive Rate (TPR) and the False Positive Rate (FPR) were calculated as:

$$TPR = {\int }_{ Y 1}^{∞} {P}_{1} ( {s}_{i} \vert x ) d x , FPR = {\int }_{Y 1}^{∞} {P}_{0} ( {\{ {s}_{j} \}}_{jTi} \vert x ) d x$$

The data shows ROC curves for different bit precisions. The optimal discriminative threshold +, was selected to maximize the area under the graph. A 10-bit precision was found to be optimal. See, FIG. 17C.

The performance of a compressive sorting module was compared with a classical spike sorting technique based on the PCA and Expectation-Maximization (EM) cluster cutting applied in the two dimensional principal component feature space. The PCA/EM method achieved 91% success rate as compared to 90% success with the presently disclosed compressive sorting module. Similar performance levels may be obtained by implementing a low pass FIR filter on the estimated coefficients to remedy the shift variance property of the DWT. Aghagolzadeh et al., “Compressed and Distributed Sensing of Neuronal Activity for Real Time Spike Train Decoding,” IEEE Transactions on Neural Systems and Rehabilitation Engineering 17: 116-127 (2009). It can be shown that the number of computations required for training the PCA/EM algorithm is in the order of O(1760m+40m²), where m is the number of training events, while the number of computations needed for the training of a compressive sorting algorithm is in the order of O(1440m+10m²). Comparing the efficiency of the two algorithms in terms of the number of computations needed to sort a fixed number of events, the compressive sorting module is approximately 4 times more efficient than the PCA/EM offline algorithm, which implies larger savings in area and power consumption.

In one example embodiment, an efficient and simple VLSI hardware architecture for real-time spike sorting with optimized size and power budgets suitable for implantable BMI systems is provided. In one embodiment, the architecture comprises a module based on sparse representation analysis of the data by means of DWT followed by smart thresholding. The data presented herein demonstrates that spike sorting is performed on compressed data. For example, spike sorting may be performed without waveform decompression and/or reconstruction. In one embodiment, the architecture comprises approximate 22K transistors using a 0.18 μm CMOS microchip. In one embodiment, the transistors comprise less than 0.1 mm² of the chip area. In one embodiment, the transistors pass through approximately 31 μW of power to process 32 channels of data at 5 levels of DWT decomposition.

Although not wishing to be bound by this proposed theory, it is believed that this module can easily transfer the maximum theoretically possible neural activity from a neural ensemble recorded by a 32-channel array without pushing the bandwidth and power limits of a transceiver. Oweiss K., “A systems approach for data compression and latency reduction in cortically controlled brain machine interfaces” IEEE Transactions on Biomedical Engineering 53:1364-1377 (2006). It is further believed that the architecture design disclosed herein results in substantial savings in computational and communication costs for implantable neural prosthetic systems.

### V. Therapeutic Applications

In one embodiment, the neural data acquisition and processing described herein may be used in research and/or clinical settings. For example, the wireless data collection and processing systems are contemplated to improve assistive technologies designed to restore sensory and motor functions lost through injury or disease by directly translating the neural signals related to movement intention in the brain to control commands that operate prosthetic limbs or computers. Alternatively, the wireless data collection and processing systems are contemplated to improve two-way BMI's (i.e., for example, output-input BMIs) that provide the ability to recognize events related to neurological disorders such as epilepsy and provide interventional treatment (i.e., for example, medical infusion and/or nerve stimulation).

A. Skeletalmuscular Conditions

BMI technology is barely 10 years old, but it has evolved very quickly. One of the first demonstrations enabled a rat to use a robotic arm to grab drops of water and move them to its mouth. Later reports demonstrated the same technology in primates. Human studies have been reported using surgically implanted BMIs in Parkinson's patients. Clinically useful BMI devices utilized closed-loop sensors that can generate feedback, to inform the brain regarding device performance. Improvements in the field of BMI may be expected to assist paraplegic or quadriplegic patient walk again. For example, the spinal cord may be by-passed and, instead, a wireless link may be used to send a message from a brain surface microchip an exoskeletal prosthetic device, which will facilitate walking. BMI allows the brain to act independently of the body. Patients will not only be able to control devices that they wear, but also operate devices that are some distance away while experiencing feedback from them.

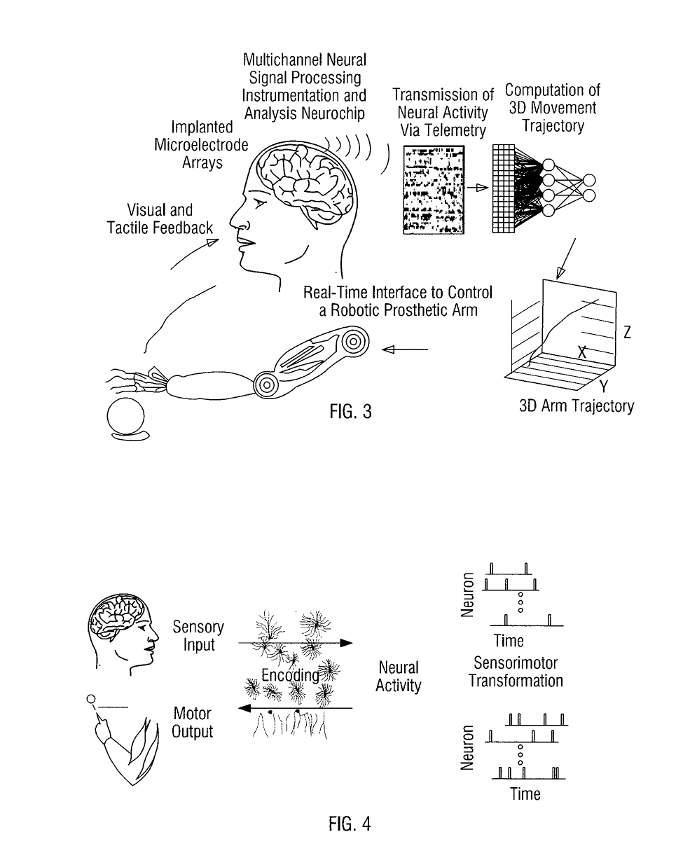

Another clinical application of HBMIs may restore different aspects of motor function in patients with severe body paralysis, caused by conditions including but not limited to, strokes, spinal cord lesions or peripheral degenerative disorders. See, FIG. 3. Multiple, chronically implanted, intracranial microelectrode arrays would be used to sample the activity of large populations of single cortical neurons simultaneously. The combined activity of these neural ensembles would then be transformed by a mathematical algorithm into continuous three-dimensional arm-trajectory signals that would be used to control the movements of a robotic prosthetic arm. A closed control loop would be established by providing the subject with both visual and tactile feedback signals generated by movement of the robotic arm. Neural signals from healthy regions of the brain could be ‘re-trained’ to control the movements of artificial prosthetic devices, such as a robotic arm. For example, paralyzed patients have been taught to use brain signals obtained from their motor cortex to interact with computers. Kennedy et al., NeuroReport 9:1707-1711 (1998).

Extensive electrophysiological work in primates and imaging studies in humans have shown that multiple interconnected cortical areas in the frontal and parietal lobes may be involved in the selection of motor commands that are believed to control the production of voluntary arm movements. Wise et al., Annu. Rev. Neurosci. 20:25-42 (1997). Although each of these areas has different degrees of functional specialization, in theory, each of them could be selected as the source of brain signals for controlling the movements of an artificial device. Within each of these cortical areas, different motor parameters, such a force and direction of movement, are coded by the distributed activity of populations of neurons, each of which is typically broadly tuned to one (or more) of these parameters. This indicates that implementations of HBMIs for robotic arm control may rely on intracranial recordings from large populations of single neurons to derive motor control signals.

100-1,000 cortical motor neurons are expected to yield sufficient multielectrode intracranial recordings to support motor control signals. For example, a precise off-line reconstruction of complex three-dimensional arm trajectories has been reported by using simple multiple regression techniques to transform the activity of 300-400 serially recorded cortical motor neurons into a neural population vector. Schwartz, A., Science 265:540-542 (1994). Moreover, rat and primate research has shown that simple, real-time algorithms, applied to samples of 50-100 simultaneously recorded cortical neurons, can be used to control robotic devices in real time and mimic three-dimensional arm reaching movements. Chapin et al., L. Nature Neurosci. 2: 664-670 (1999); and Wessberg et al., Nature 408:361-365 (2000), respectively.

To achieve seamless interactions with prosthetic devices, patients should receive sensory feedback information (i.e., for example, visual or tactile signals) from a prosthetic limb. These feedback signals will establish a closed control loop between the brain and artificial devices and will probably help patients learn how to operate HBMIs. Studies in rats have revealed that when visual feedback information coupled with reward for a successful movement of a robotic limb, the rats progressively ceased to produce corresponding natural limb movements. Chapin et al., L. Nature Neurosci. 2: 664-670 (1999). In other words, even though the rats continued to exhibit the patterns of cortical activity reflective of natural limb movements, no significant natural limb movement occurred. This indicates that motor control signals can be generated by cortical neurons without any muscle activity, and hence that paralyzed patients might be capable of learning to operate a robotic arm even though they cannot move their own limbs.

These observations also raise the intriguing hypothesis that, by establishing a closed control loop with a BMI, the brain could incorporate electronic, mechanical or even virtual objects into its somatic and motor representations, and operate upon them as if they were simple extensions of our own bodies. The adult cortex is capable of significant functional reorganization (or plasticity) after events including but not limited to: i) peripheral and central injuries (Wu et al., J. Neurosci. 19:7679-7697 (1999)); ii) changes in sensory experience (Polley et al., Neuron 24:623-637 (1999)); and iii) learning of new motor skills (Laubach et al., Nature 405:567-571 (2000)).

Indeed, the notion that adult plasticity can dynamically alter the perception of the limits of our own body is corroborated by studies on patients who have undergone limb amputations. Immediately after the amputation, most of these patients experience the sensation that their amputated limb is still present and moving. These ‘phantom limb’ sensations are paralleled by a significant plasticity of body maps in the somatosensory cortex, the part of the brain that receives and interprets sensory signals from areas such as the skin surface. Ramachandran V. S., Proc. Natl. Acad. Sci. USA 90:10413-10420 (1993). Instead of remaining silent, the areas in these brain maps that used to represent the amputated limb progressively start to respond to stimulation of neighboring body regions spared by the amputation. Thus, it is conceivable that tactile feedback signals, generated by the movements of a brain-controlled robotic arm and delivered to the patient's skin, could be used to incorporate the representation of such an artificial device into cortical and subcortical somatotopic maps.

Other reports have suggested that neural implants not only translate brain signals into movement, but also evolve with the brain as it learns. Instead of simply interpreting brain signals to help paralyzed patients and amputees control prosthetic limbs with just their thoughts, these BMIs would adapt to a person's behavior over time, and use the knowledge to help him/her complete a task more efficiently. “New prototype neural implant learns with the brain” Hindustan Times (Jun. 25, 2008). At present, the reported data is limited to the brain doing all the talking and the machine following commands.

One model BMI-learning system is based on setting goals and giving rewards. During one study, electrodes were implanted into rat brains wherein the captured signals were transmitted to a computer. The rats were taught to move a robotic arm towards a target with just their thoughts. using a water drop as a reward. The computer was programmed to facilitate the training by earning points whenever the rat moved the arm closer to the target.

This computer program resulted in a more efficient process to determine which brain signals lead to the most rewards.

B. Remote Cognition

It has been reported that neuronal signals from a monkey, trained to walk upright on a treadmill, remotely controlled the walking of a robot, located more than 10,000 km away. “Technical Innovation At The Brain-Machine Interface” Nikkei English News (Oct. 9 2008). Such experiments might lead to the development of technologies for rescue robots and self-controlled prosthetic legs. Further, computer operations can also be performed using mental intentions of action. Although not wishing to be bound by this proposed theory, it is believed that when a person focuses their attention or moves their body, discernible changes take place in brainwave and blood flow patterns in the brain. It is this kind of data that can be monitored to discern a person's intentions and translate them into machine-directable commands.

One clinical BMI trial involved two patients have been implanted the BrainGate Neural Interface System (Cyberkinetics Neurotechnology Systems). This trial evaluated patients with quadriplegia due to spinal cord injury, stroke or muscular dystrophy for a period of 12 months. Interim results showed that at least on patient used the system to control a computer using thoughts. The BrainGate Neural Interface System is a proprietary, investigational brain-computer interface that consists of an internal sensor to detect brain cell activity and external processors that convert these brain signals into a computer-mediated output under the person's own control. “Cyberkinetics Provides Update on BrainGate Clinical Trial” Wireless News (4 Apr. 2005). The BrainGate sensor is a tiny silicone chip about the size of a baby aspirin with one hundred electrodes, each thinner than a hair, that detect the electrical activity of neurons. The sensor is implanted on the surface of the area of the brain responsible for movement, the primary motor cortex. A small wire connects the sensor to a pedestal, which extends through the scalp. An external cable connects the pedestal to a cart containing computers, signal processors and monitors, which enable the study operators to determine how well a study participant can control his neural output. Two primary goals of the BrainGate study was to characterize the safety profile of the device and to evaluate the quality, type, and usefulness of neural output control that patients can achieve using thoughts. The sensor portion of the BrainGate neural interface is surgically implanted into the area of the brain responsible for movement. Performance tasks with the device include controlling the movement of a cursor on a screen toward a specific target with their thoughts. The study is expected to last for about 12 months for each patient. At the end of the study, each participant will undergo another surgery to have the device removed or may have the option to participate in future studies, the company noted in a release.

C. Brain Mapping

Brain surgery is driven by new and unforeseen technologies involving surgical innovations, device implants, and/or neural prostheses. Despite the current limitations of each—for example, optical devices do not yet exist—the approaches detailed in the following pages are at the center of newfound interest in the brain. Operating on an organ as complex and fragile as the brain to remove a tumor or limit the spread of epileptic seizures from one part of the brain to another poses a challenge that is simple to describe, yet hard to address. One problem is how to precisely define which tissue is to be removed and which tissue should not be removed. “Mechanical minds: New surgical methods, devices, and research efforts could revolutionize the treatment of brain disorders” Red Herring (1 Oct. 2001)

Such techniques require good imaging and fine navigation regarding both the target and the angle of approach. In the past century, brain imaging has evolved from X rays to high-resolution computed tomography and magnetic resonance imaging (MRI). Functional MRIs help identify specific brain regions involved in particular activities. Still, when a surgeon opens a patient's head, he essentially operates by dead reckoning, a situation that the rapidly growing field of image-guided surgery is now changing.

3D brain mapping systems are becoming available (i.e., for example, StealthStation Medtronic). At the start of surgery, light emitting diodes or electromagnetic sensors are attached to the surgical instruments and the brain as markers. During surgery, the system matches the instrument position to the 3D map and displays it on a computer screen, enabling the surgeon to see the critical area within a millimeter of accuracy. However, 3D maps have significant drawbacks because even thought the 3D maps are still created with historical images, taken hours before the operation as soon as you open the brain, the orientation changes. For example, if the surgical procedure excises a tumor, the surrounding tissue may collapse into the void, thereby altering the brain's structural orientations.

D. Deep Brain Stimulation

Alternatives to brain surgery encompass methods for therapeutic brain stimulation (i.e., for example, deep brain stimulation, DBS). A DBS device is similar to a cardiac pacemaker in that is implanted beneath the skin near the collarbone. Subcutaneous leads snake up through a small hole in the skull and activate electrodes in the target brain structure. Patients trigger the device by passing a small magnet over the implant. DBSs have been used to treat Parkinson's disease and essential tremor, and/or other movement disorders otherwise imperfectly controlled by medication or surgery. See, Table 3.

TABLE 3
Estimated Neuronal Disorder Patient Number In The United States¹

| Disorder | Estimated Patient Number |
|---|---|
| Alzheimer's disease | 400,0000 |
| Stroke | 300,0000-400,0000 |
| Traumatic brain injury | 2,500,000-3,700,000 |
| Epilepsy | 1,750,000 |
| Parkinson's disease | 1,500,000 |
| Multiple sclerosis | 250,000-350,000 |
| HIV (AIDS) dementia | 60,500-157,300 |
| Amyotrophic lateral sclerosis | 30,000 |
| Huntington's disease | 30,000 |
| Brain tumor | N/A |
| TOTAL PREVALENCE | 13,120,500-15,517,300 |

¹Family Caregiver Alliance.

Alzheimer's disease and stroke are the most prevalent causes of adult-onset brain impairment in United States. DBS is also being used to treat epilepsy where patients can use the implant to short-circuit a generalized seizure upon encountering a prodromal syndrome. Improvements to these systems may involve a closed-loop system in which a detection device monitors brain activity for the characteristic signature of an impending seizure, and then automatically either triggers a DBS pulse or infuses small doses of a drug through an implanted cannula—a tube similar to a catheter. Problems remain in refining the system's detection algorithms so that impending seizures won't be missed and treatment will only be given when necessary.

E. Auditory Implants

Deafness has been treated by use of cochlear implants that consist of a microphone, a speech-processing device, and electrode arrays that transmit information to the auditory nerves, bypassing damaged biological structures. Because different portions of the normal cochlea resonate at different frequencies transducer cells inside the cochlea may translate positional information into signals representing different pitches. The implants produce sound upon stimulation, but patients must learn to interpret the information. Over a period of months, the brain learns to interpret the input as intelligible sound and eventually even music. Similarly, visual neural prostheses may eventually result in an artificial retina

F. Epilepsy

Estimates indicate that about 0.5-2.0% of the population has epilepsy. McNamara, J. O., Nature 399(Suppl.), A15-A22 (1999). About 10-50% of these patients do not respond well to current antiepileptic medications and may not be candidates for surgery. Throughout this century, multichannel recordings from scalp, brain surfaces and even chronically implanted intracranial electrodes have been used to investigate the electrophysiological activity that characterizes different types of seizure in humans. By doing so, different types of epilepsy have been identified and distinct patterns of neurophysiological activity are associated with the initiation and establishment of a seizure attack. Epilepsy research indicates that the development of an unsupervised HBMI for monitoring, detecting and treating seizure activity may have clinical applicability. See, FIG. 2A.

For certain types of seizure, there seems to be a particular spatiotemporal pattern of cortical activity that appears seconds or even minutes before the full epileptic attack starts. Recent reports have suggested that automatic seizure-prediction algorithms can be applied to intracranial and scalp recordings to forecast the occurrence of a seizure. Martinerie et al., Nature Med. 4:1173-1176 (1998); and Webber et al., Electroencephalogr. Clin. Neurophysiol. 98: 250-272 (1996). Such seizure-prediction algorithms might provide sufficient time (i.e., for example, 2-5 minutes) to warn the patient of an imminent attack, and to trigger automatic therapeutic intervention (i.e., for example, anti-epileptic medication release) before convulsion or loss of consciousness. However, not all patients are responsive to anti-epileptic medications.

Animal and human subject research has revealed that electrical stimulation of peripheral cranial nerves, such as the vagus and trigeminal nerves, can substantially reduce cortical epileptic activity. Zabara, J., Epilepsia 33:1005-1012 (1992); and Fanselow et al., J. Neurosci. 20: 8160-8168 (2000). This peripheral nerve stimulation may be applied before the initiation of seizure or during its initial stages, such that a significantly higher reduction of seizure activity can be achieved. Such a device could be coined a ‘brain pacemaker’ and would rely on arrays of chronically implanted electrodes to search continuously for spatiotemporal patterns of cortical activity indicating an imminent epileptic attack. See, FIG. 2A. Instrumentation neurochips would be responsible for all the basic signal-processing operations. They would also provide signals to one or more seizure-prediction algorithms, implemented into analytical neurochips, which would carry out real-time analysis of cortical activity. Once pre-seizure activity patterns were detected, the analytical neurochip could trigger electrical stimulation of one or multiple cranial nerves. In patients who respond to pharmacological therapy, the same stimulator could be used to activate a minipump to deliver one or more anti-epileptic drugs directly into the blood stream. A simplified implementation of this concept has been used successfully in rats. Fanselow et al., J. Neurosci. 20: 8160-8168 (2000).

### Wireless Communication of Neural Data

Long-term continuous intracortical recording of neuronal ensembles in freely behaving subjects requires a reliable wireless communication channel for transmitting important biological information. The need for ultra low-power, fully implanted recording systems, however, make the design of the wireless transmission protocol more demanding. Here, Applicants introduce an adaptive protocol that can cope with the variable characteristics of the errors in the wireless channel associated with different levels of subject mobility, for example, during rest and active states. The wireless channel is modeled as a finite-state Markov channel, in which states are binary symmetric channels with different binary error rates. A convolutional encoder with a specific code rate is incorporated into each state, for which the length of data transmission packets is optimally estimated. The protocol can switch between different states depending on subject mobility to ensure a highly reliable communication channel, while optimizing the power consumption by minimizing the average memory length required for storing packets prior to transmission.

### I. INTRODUCTION

Spike trains are the fundamental communication means through which neurons transmit and process information in the nervous system. Understanding how information is processed in the brain by means of spike trains is a fundamental goal in systems neuroscience in order to better understand the complex mechanisms underlying brain functions in the normal and diseased states.

To measure the spike train activity of multiple neurons simultaneously, microelectrode arrays have to be implanted in the brain for prolonged periods of time. Because these arrays record a mixture of spiking activity from populations of neurons in the vicinity of the electrodes, spike sorting is needed to segregate the activity of each recorded cell. This requires transmitting the high bandwidth neural data through a wired connection to an external computer to perform this task before any biologically relevant information can be extracted and interpreted.

Wireless transmission of ensemble neural activity is highly desirable, both in basic and clinical neuroscience applications. This is because tethering the subject to the recording system limits the scope of experiments that can be designed. For clinically viable Brain Machine Interfaces, fully implanted systems with wireless communication capability minimize any potential risk of infection and discomfort to the subject while elongating the implant's lifespan.

While typical wireless communication applications necessitate low-power communication protocols to be used, they do not put strict constraints on other hardware resources, such as the memory size required to store packets prior to transmission, or the number of transmission requests. In this paper, Applicants propose a new protocol for wireless transmission of neural data that simultaneously minimizes the power and size requirements of the implant. This is achieved by optimizing the data packet length and minimizing the number of service requests based on the behavioral state of the subject. This approach substantially reduces the service time.

### II. THEORY

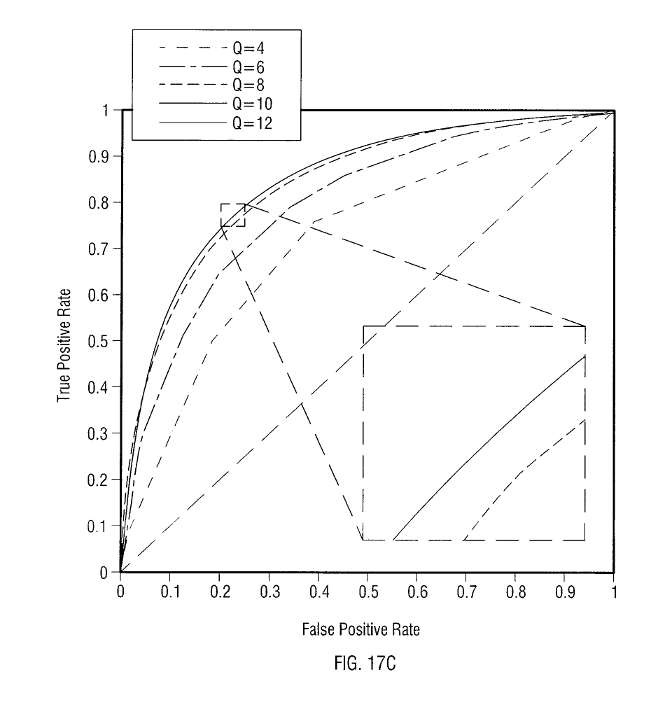

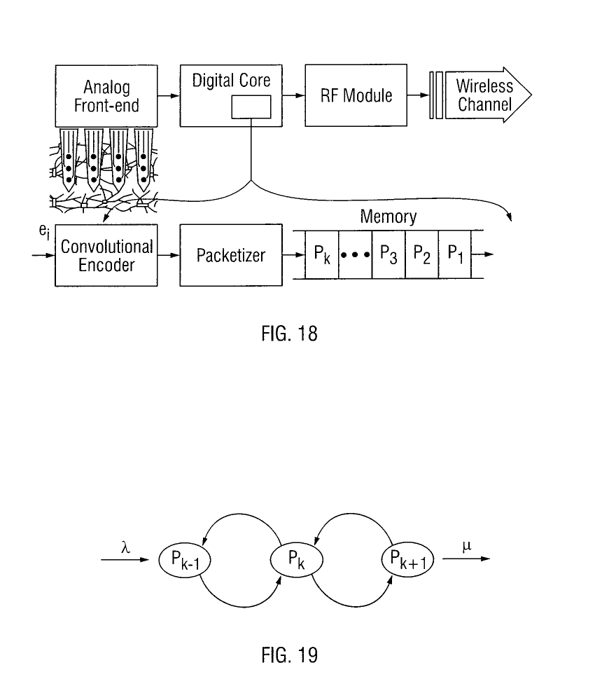

FIG. 18 demonstrates the implantable wireless transmission module. The digital core is a neuro-processor that provides the spike events, { [symbol] 56 ={ [symbol] . . . }. Each event, [symbol] ᵢ, contains information about the specific neuron from which the spike was detected and the relative time of the spike firing. These events are coded by a convolutional encoder and then packetized with a certain length, along with start and end sequences. Convolutional codes are a type of error correcting codes that can detect and correct errors within a certain limit using other transmitted digits. Packets in these codes are queued in a memory block prior to transmission to prevent loss of data, especially when the channel is busy. Once the channel is free, packets can be transmitted in the order they were received (first-in first-out).

### A. Birth-Death Process

Assume that the data packets arriving at time {t [symbol] }{ [symbol] . . . , [symbol] } can be modeled as a Poisson process. In this model, the number of packets residing in the memory, k, can be used to determine the current state of the memory, pₖ. The transitions between the different states in this model follow a birth-death process, in which the state, pₖ, can transit to either pₖ₋₁ when a packet is serviced out of the queue, or pₖ₊₁ when a new packet joins the queue, as shown in FIG. 19. Let us assume that the service time for each packet, { [symbol] }{ [symbol] ={ [symbol] . . . , [symbol] _{T}}, follows a uniform or exponential distribution. In queuing theory, such model can be characterized by the mean arrival rate, λ, and the mean service rate, μ, both measured in bits per second.

In a queue at equilibrium, the average number of packets in the memory is L=λ/(μ−λ), and its variance is L+L [symbol] . Considering that the size and power of the internal memory of the implanted system is limited, a primary goal is to minimize L, which in turn requires identifying the key factors contributing to the mean arrival and mean service rates.

The mean arrival rate, λ, depends on the level of the activity of the recorded neural ensemble. It can be factored as the product of the number of packets sent per time unit f, and the length of a packet in bits, N, as λ=Nf. However, in some example embodiments, changing N may appropriately change f to cope with the instantaneous rate.

The mean service rate, μ, on the other hand, is a product of the available channel capacity, [symbol] , the data overhead, δ, and the probability of accepting a packet, P [symbol] . The data overhead, 0<δ<1, is a redundancy factor introduced by the convolutional encoder and the packetizing unit. By encoding, each m-bit symbol is transformed into an n-bit symbol, where r=m/n is the code rate.

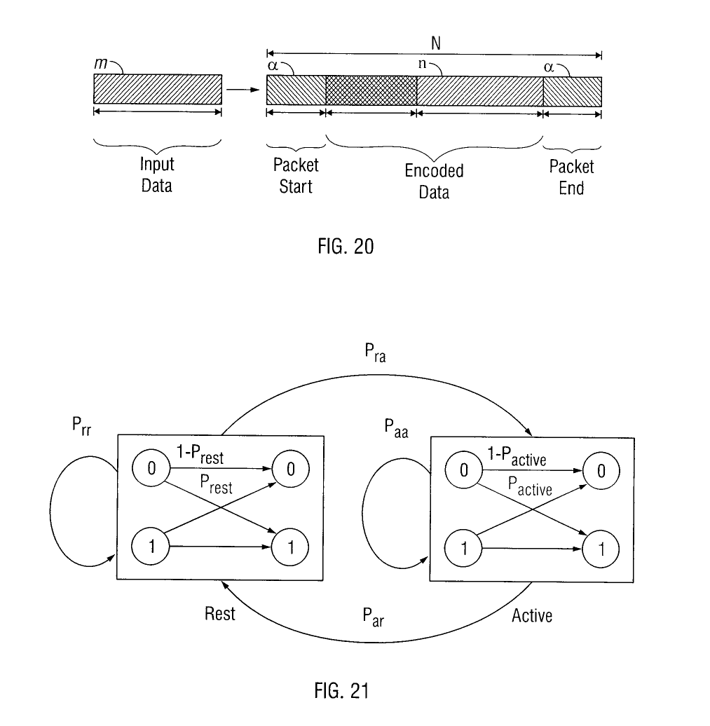

It can be seen from FIG. 20 that the overhead introduced after encoding and packetizing is [symbol] = [symbol] r/( [symbol] +2ar), in which 2α is the additional packet length introduced by making the packet's start and end sequences. It can be simply shown that the overhead [symbol] =(N−2α)r/N is only a function of the packet length, N. Replacing the independent factors in L, the average memory length can be expressed as

$$L = \frac{λ N}{{P}_{a} B ( N - 2 a ) r - λ N} \qquad ( 1 )$$

Except for B, α, r and λ, which are fixed by design, the factors P [symbol] and N can be optimized to minimize the memory length.

### B. Channel Model

A major source of errors in the wireless channel is due to the noise caused by subject's movement. Because our design relies on an inductive data transmission link, any potential misalignment between the data telemetry coupling coils could cause erroneous data transmission. In some example embodiment, to characterize P [symbol] under this type of error, the wireless channel may be first modeled to characterize the process under which errors occur.

Let's assume that the channel at any time point can be modeled as a binary symmetric channel (BSC) with a particular binary error rate (BER), ρ. In a BSC, the transmitter sends a bit (a zero or one), in which the probability that this bit will be flipped (zero to one or one to zero) is equal to p. Such a channel can be modeled as a Markov process that switches between different states of operation, known as the finite-state Markov channel (FSMC) [6]. Therefore, states of operation for the FSMC can be obtained by categorizing the subject's behavior into different levels of mobility, such as rest and active states. In such case, errors for these states can be characterized by ρₑₛₜ and ρ [symbol] of the BSC, respectively, as shown in FIG. 21.

The error correction capability of the convolutional code is determined by the error correction ratio, α. A decoder can correct up to |αN┘ number of errors for a packet with length N, where |x| is the maximum integer number smaller than x. Therefore, P [symbol] can be estimated as

$${P}_{a} = \text{?} \text{?} {( 1 - ρ )}^{x - l} ( \begin{matrix}N \\ i\end{matrix} ) \\ \text{?} \text{indicates text missing or illegible when filed} \qquad ( 2 )$$

It can be seen from (2) that P [symbol] only depends on the packet length, N. Therefore, the average length of the internal memory can be expressed as a function of the variable N.

### III. RESULTS

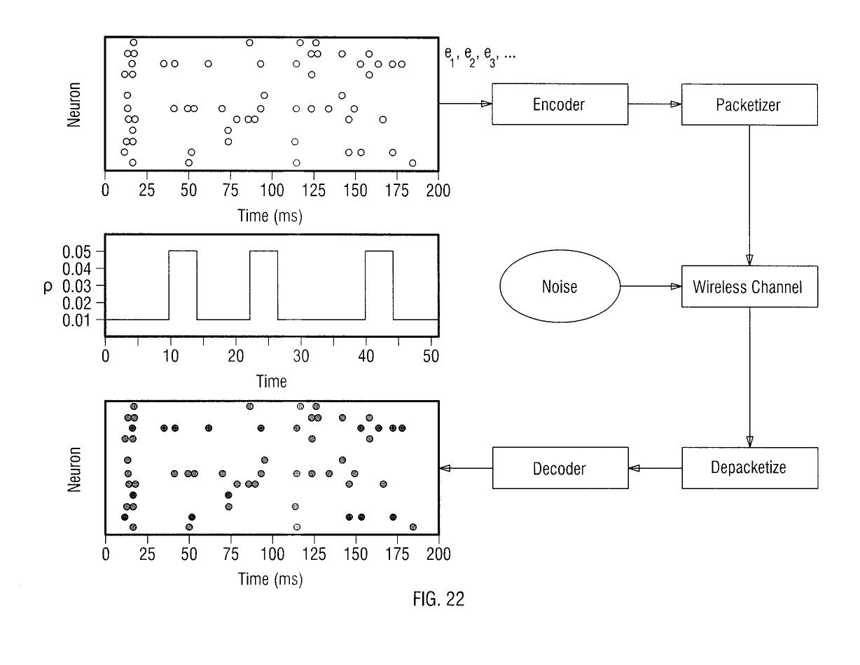

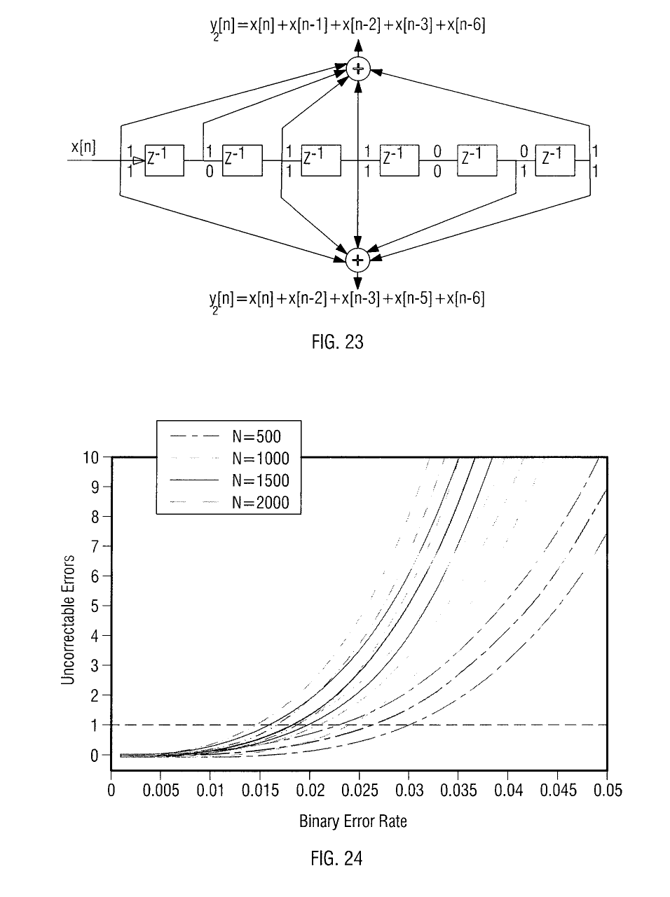

In some example embodiment, to characterize the effects of changing the packet length on the memory length, a noisy wireless channel with a time-varying binary error rate, ρ, may be simulated as shown in FIG. 22. The input data stream contains detected spike events from in-vivo recordings in the barrel cortex of an anesthetized rat. These events were streamed into a 7^{th}-order convolutional encoder, as shown in FIG. 23. This encoder has two output data streams, determined by their individual generating functions, thus, providing a data rate of 0.5.

In some example embodiments, to determine the relationship between the error correction capability of the decoder and the packet length, to 10 [symbol] packets may be introduced to the wireless channel. The variable error rate was applied to the noisy channel by randomly varying ρ between 0 and 0.1. Since in this case the input data stream is known, the maximum number of errors that a decoder was able to correct may be estimated. FIG. 24 demonstrates the average number of uncorrected errors versus the BER, ρ, for different packet lengths. In one example embodiment, by setting the number of acceptable uncorrected errors to one, as shown by the dotted line in FIG. 24, the maximum number of correctable errors for a packet length and therefore, estimate |αN. may be determined.

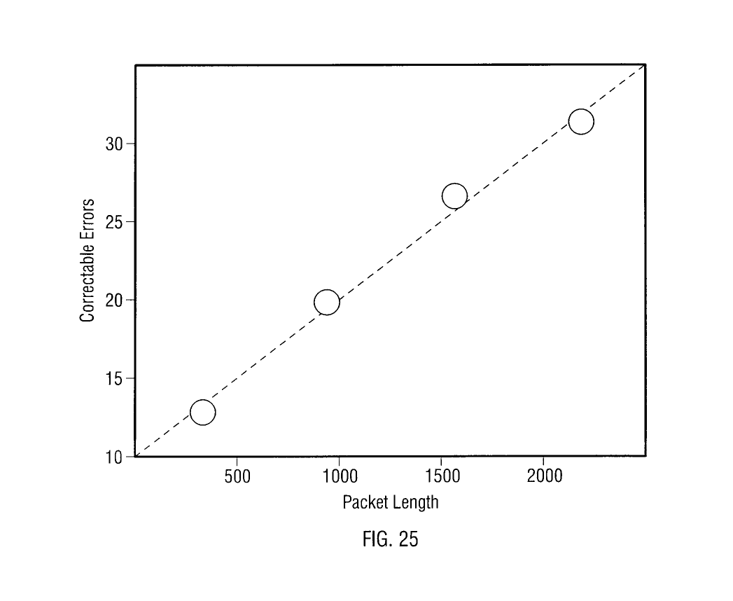

FIG. 25 demonstrates the maximum number of correctable errors, |αN, versus the packet length, N. Interestingly, this relation can be linearly modeled as |αN┘=0.0188×N+7. By substituting in equations (1) and (2), the average memory length in bits is estimated as

$$G ( N ) = \frac{λ {N}^{2}}{B ( N - 2 α ) r \text{?} \text{?} {( 1 - ρ )}^{N - L} ( \begin{matrix}N \\ ɛ\end{matrix} ) - λ N} \\ \text{?} \text{indicates text missing or illegible when filed} \qquad ( 3 )$$

Therefore, the memory length, [symbol] (N), is only a function of the packet length, N, while the rest of the variables are design parameters.

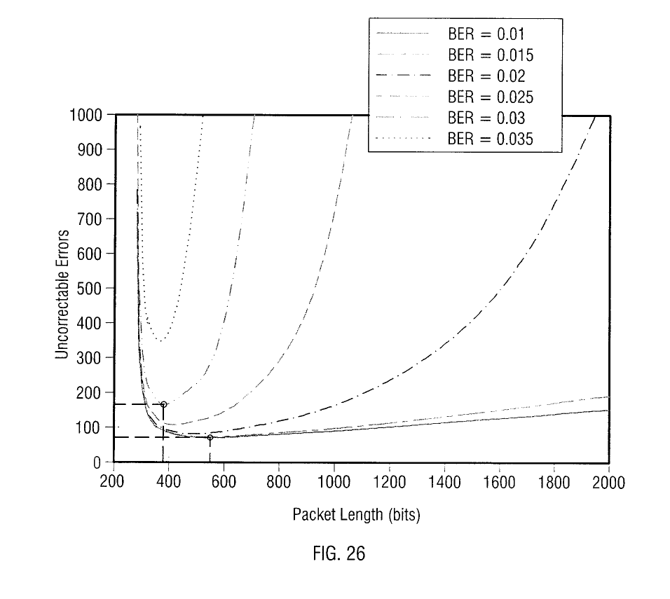

Using (3), FIG. 26 illustrates the average memory length versus packet length for various BER. It can be seen from FIG. 26 that the optimal packet length varies for different BER. For example, the optimal packet length for [symbol] =0.01 is 550 bits, while it is 380 for [symbol] =0.08. Note that these plots are obtained for the 7^{th}-order encoder illustrated in FIG. 23, and changing the encoder type will produce different optimal packet lengths.

As illustrated by the square wave in FIG. 22, the activity of the subject, and accordingly the associated BERs, was modeled by two levels of mobility, the rest and active states. Since ρₑₛₜ is smaller than ρ_{active}, a convolutional encoder with a higher rate is suggested for the rest state, such as ⅔. To find the optimal packet length for this state, the procedure from FIG. 24 to FIG. 26 is repeated.

Switching between different states of mobility can be done by the transmitter using an accelerometer that is mounted on the implantable system. Once the level of mobility, captured by the accelerometer, exceeds some threshold, the transmitter switches to a convolutional encoder with a lower rate to increase the error correction capability and thus to increase the probability of accepting the transmitted packets. It is noteworthy that the model presented here assumes the minimum number of states, and is certainly the simplest. More states can be included in future system design.

### IV. CONCLUSIONS

Applicants presented an adaptive wireless communication protocol for reliable transmission of intracortical neural recordings in freely behaving subjects. Applicants suggested using convolutional encoders to provide the receiver with the ability to correct errors that occur due to the noisy channel. The encoded data stream is packetized and stored in a memory block prior to transmission.

In some example embodiments, to determine the optimal memory block size, Applicants modeled the queue of packets in the memory block as a birth-death process and estimated the average memory length, L. Applicants derived a closed form for L as a function of the packet length, N, and used it to minimize the required memory length. Also, in some example embodiments, to incorporate the variable characteristics of the error process relative to the subject's activity, the wireless channel may be modeled as a finite-state Markov channel, with rest and active states. In this model, each state has a particular code rate, and accordingly a specific packet length. Switching between different states is controlled by the transmitter, which continuously monitors the subject's mobility.

The proposed wireless communication protocol meets the requirements of a low-power, small-size implantable system through two key design features: 1) the power consumption is reduced by limiting the total number of transmissions through increasing the success rate of each transmission, thereby reducing the service time; 2) the system size is reduced by optimizing the packet length to consume the least amount of memory, which also results in additional savings in power consumption.

In some example embodiments, channel errors in the case of sparsely represented neural data during wireless transmission may be more costly than errors in the case of transmission of uncompressed raw data. Since our current design uses a half duplex channel, the proposed protocol will further enable replacing inconvenient handshaking mechanisms. In some example embodiments, the proposed protocol may be used in other BMI applications with unreliable and time varying wireless communication channel that may be encountered during a myriad of behavioral states in a freely moving subject.

### Machine-Readable Media, Methods, Apparatus, and Systems

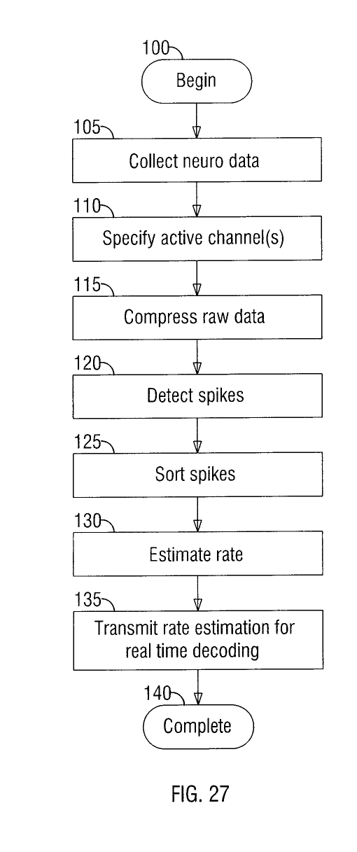

FIG. 27 is a flow diagram of various methods according to some example embodiments. The methods may include the following actions:

at block 100, the methods may begin;

at block 105, neuro data from an organ, such as a brain, may be collected;

at block 110, raw data may be sequentially passed through and at least one active channel may be specified;

at block 115, raw data may be compressed and transmitted for offline analysis;

at block 120, spikes may be detected;

at block 125, the spikes may be sorted;

at block 130, a underlying neuronal firing rates may be estimated;
at block 135, the estimated rates may be transmitted, wired or wirelessly, outside the organ for instantaneous decoding; and

at block 140, the methods may terminate.

The methods described herein do not have to be executed in the order described, or in any particular order, unless so specified. Moreover, various activities described with respect to the methods identified herein can be executed in repetitive, looped, serial, or parallel fashion. The individual activities shown in the methods described herein can also be combined with each other and/or substituted, one for another, in various ways. Information, including parameters, commands, operands, and other data, can be sent and received in the form of one or more carrier waves.

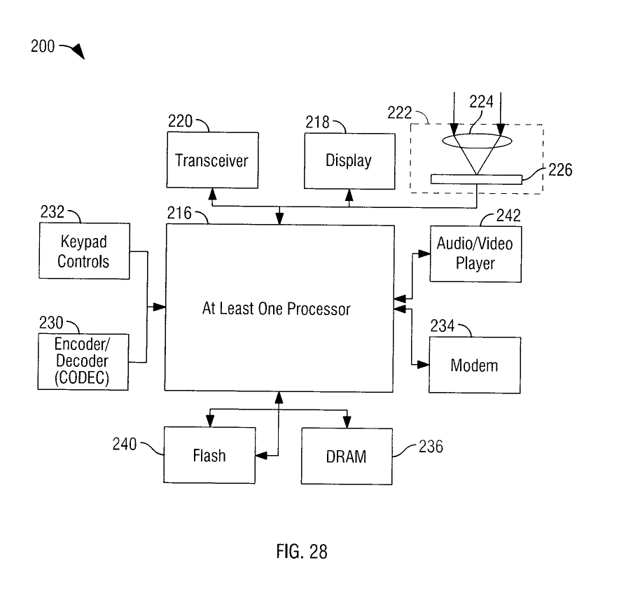

FIG. 28 is a block diagram of a system 200 according to various example embodiments. The system 200 may include one or more apparatus, such as an encoder/decoder (codec) 230. The system 200, in some embodiments, may comprise at least one processor 216 coupled to a display 218 to display data processed by the at least one processor 216. The system 200 may also include a wireless transceiver 220 (e.g., a cellular telephone transceiver) to receive and transmit data processed by the at least one processor 216. In various embodiments, the system 200 may comprise a modem 234 coupled to the at least one processor 216.

The memory system(s) included in the apparatus 200 may include dynamic random access memory (DRAM) 236 and non-volatile flash memory 240 coupled to the at least one processor 216. In various embodiments, the system 200 may comprise a camera 222, including a lens 224 and an imaging plane 226 coupled to the at least one processor 216. The imaging plane 226 may be used to receive light rays 228 captured by the lens 224. Images captured by the lens 224, including images of an organ, such as a brain, may be stored in the DRAM 836 and the flash memory 240. The lens 224 may comprise a wide angle lens for collecting a large field of view into a relatively small imaging plane 226. In many embodiments, the camera 222 may contain an imaging plane 226.

Many variations of system 200 are possible. For example, in various embodiments, the system 200 may comprise an audio/video media player 242, including a set of media playback controls 232, coupled to the at least one processor 216. Although shown as separate apparatus in FIG. 2, the encoder/decoder (codec) 230 may be provided as part of the audio/video media player 242 in some example embodiments. The apparatus in the system 200, such as the at least one processor 216 and the encoder/decoder (codec) 230, may be used to implement, among other things, the processing associated with the methods 100 of FIG. 1. The at least one processor 216 may be a general processor or an application specific processor or any other suitable processors.

In one example embodiment, at least one of the apparatus in the system 200 may include one or more modules. For example, the system 200 may comprise a neuroprocessor unit, such as a Neural Interface Node (NIN) described in FIG. 15A. In one example embodiment, the NIN module may comprise multiple electrode arrays (MEAs), an amplifier/filter, an A/D converter, a first multiplexer, a discrete wavelet transform (DWT), at least one threshold module, such as a channel threshold module or a node threshold module, a run length encoder, a compressive spike sorting module (not shown in FIG. 15A), second multiplexer, a packetizer, a power manager, a data/power transceiver, and a clock generator.

In one example embodiment, the data/power transceiver may comprise two separate orthogonal coils for power and data with two different carrier frequencies. In one example embodiment out diameter may be 10 mm and substrate thickness may be 1.5 mm. Different frequencies may be supported by the data/power transceiver, such as 5 MHz, 10 MHz or 13.56 MHz.

In one example embodiment, the system 200 may comprise a Manager Interface Module (MIM) processor described in FIG. 15B. In one example embodiment, the MIM processor may comprise a CRC check, a signal modulator, a power source, a power amplifier, a power manager, a power transceiver, a CBS transceiver, a data transceiver, a packetizer (not shown in FIG. 15B), a run length decoder (not shown in FIG. 15B), a EDWT (not shown in FIG. 15B) and a translation algorithm module (not shown in FIG. 15B). In one example embodiment the power transceiver and the data transceiver may be combined as a single entity, such as the data/power transceiver described in relation with the neuroprocessor above.

Also, any one or more of various variations of the encoder/decoder (codec) 230 may be used to implement the processing associated with the methods 100 of FIG. 1. In some example embodiments, the encoder/decoder (codec) 230 may be implemented as two separate modules: an encoder and a decoder. The encoder may be installed in a system that encodes data signals, such as neuro data collected from a brain via, for example, at least one of the multiple electrode arrays (MEAs), and transmits the encoded data signals while the decoder may be installed in another system that receives the encoded data signals and decodes them into the original data signals. In one example embodiment, the separate encoder and decoder may be installed and operated in the same system, such as the system 200, performing the same functions as those performed by a combined codec, such as the encoder/decoder (codec) 230.

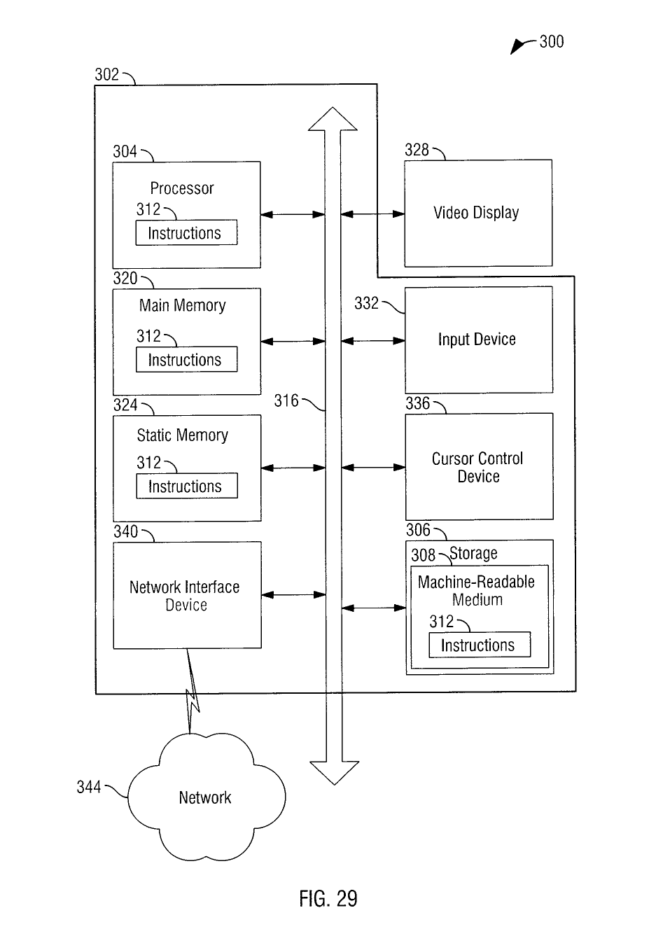

It is noted that each of the modules described herein may comprise hardware, software, and firmware, or any combination of these. Additional embodiments may be realized. For example, FIG. 29 is a block diagram of an article 300 of manufacture, including a specific machine 302, according to various example embodiments. Upon reading and comprehending the content of this disclosure, one of ordinary skill in the art will understand the manner in which a software program can be launched from a computer-readable medium in a computer-based system to execute the functions defined in the software program.

One of ordinary skill in the art will further understand the various programming languages that may be employed to create one or more software programs designed to implement and perform the methods disclosed herein. The programs may be structured in an object-oriented format using an object-oriented language such as Java or C++. Alternatively, the programs can be structured in a procedure-oriented format using a procedural language, such as assembly or C. The software components may communicate using any of a number of mechanisms well known to those of ordinary skill in the art, such as application program interfaces or interprocess communication techniques, including remote procedure calls. The teachings of various embodiments are not limited to any particular programming language or environment. Thus, other embodiments may be realized.

For example, an article 300 of manufacture, such as a computer, a memory system, a magnetic or optical disk, some other storage device, and/or any type of electronic device or system may include one or more processors 304 coupled to a machine-readable medium 308 such as a memory (e.g., removable storage media, as well as any memory including an electrical, optical, or electromagnetic conductor) having instructions 312 stored thereon (e.g., computer program instructions), which when executed by the one or more processors 304 result in the machine 302 performing any of the actions described with respect to the methods above.

The machine 302 may take the form of a specific computer system having a processor 304 coupled to a number of components directly, and/or using a bus 316. Thus, the machine 302 may be similar to or identical to the system 200 shown in FIGS. 15A and/or 15B.

Turning now to FIG. 3, it can be seen that the components of the machine 302 may include main memory 320, static or non-volatile memory 324, and mass storage 306. Other components coupled to the processor 304 may include an input device 332, such as a keyboard, or a cursor control device 336, such as a mouse. An output device 328, such as a video display, may be located apart from the machine 302 (as shown), or made as an integral part of the machine 302.

A network interface device 340 to couple the processor 304 and other components to a network 344 may also be coupled to the bus 316. The instructions 312 may be transmitted or received over the network 344 via the network interface device 340 utilizing any one of a number of well-known transfer protocols (e.g., HyperText Transfer Protocol and/or Transmission Control Protocol). Any of these elements coupled to the bus 316 may be absent, present singly, or present in plural numbers, depending on the specific embodiment to be realized.

The processor 304, the memories 320, 324, and the storage device 306 may each include instructions 312 which, when executed, cause the machine 302 to perform any one or more of the methods described herein. In some embodiments, the machine 302 operates as a standalone device or may be connected (e.g., networked) to other machines. In a networked environment, the machine 302 may operate in the capacity of a server or a client machine in server-client network environment, or as a peer machine in a peer-to-peer (or distributed) network environment.

The machine 302 may comprise a personal computer (PC), a tablet PC, a set-top box (STB), a PDA, a cellular telephone, a web appliance, a network router, switch or bridge, server, client, or any specific machine capable of executing a set of instructions (sequential or otherwise) that direct actions to be taken by that machine to implement the methods and functions described herein. Further, while only a single machine 302 is illustrated, the term “machine” shall also be taken to include any collection of machines that individually or jointly execute a set (or multiple sets) of instructions to perform any one or more of the methodologies discussed herein.

While the machine-readable medium 308 is shown as a single medium, the term “machine-readable medium” should be taken to include a single medium or multiple media (e.g., a centralized or distributed database, and/or associated caches and servers, and or a variety of storage media, such as the registers of the processor 304, memories 320, 324, and the storage device 306 that store the one or more sets of instructions 312). The term “machine-readable medium” shall also be taken to include any medium that is capable of storing, encoding or carrying a set of instructions for execution by the machine and that cause the machine 302 to perform any one or more of the methodologies of the embodiments described herein, or that is capable of storing, encoding or carrying data structures utilized by or associated with such a set of instructions. The terms “machine-readable medium” or “computer-readable medium” shall accordingly be taken to include tangible media, such as solid-state memories and optical and magnetic media.

All publications, patents and patent documents are incorporated by reference herein, each in their entirety, as though individually incorporated by reference. In the case of any inconsistencies, the present disclosure, including any definitions therein, will prevail.

Although specific embodiments have been illustrated and described herein, it will be appreciated by those of ordinary skill in the art that any arrangement that is calculated to achieve the same purpose may be substituted for the specific embodiment shown. This application is intended to cover any adaptations or variations of the present subject matter. For example, various embodiments may be implemented as a stand-alone application (e.g., without any network capabilities), a client-server application or a peer-to-peer (or distributed) application. Embodiments may also, for example, be deployed by Software-as-a-Service (SaaS), an Application Service Provider (ASP), or utility computing providers, in addition to being sold or licensed via traditional channels. Therefore, it is manifestly intended that the embodiments described herein be limited only by the claims and the equivalents thereof.

What is claimed is:

1. A device comprising a biocompatible microchip comprising a compressive spike sorting module and a transmitter, wherein the microchip is electronically connected to the transmitter.

2. The device of claim 1, wherein said microchip comprises a plurality of micro electrodes.

3. The device of claim 1, wherein said compressive spike sorting module comprises a discrete wavelet transform block.

4. The device of claim 1, wherein said compressive spike sorting module comprises a thresholding block.

5. The device of claim 1, wherein said compressive spike sorting module comprises a packet formatter block.

6. The device of claim 1, wherein said electronic connection between said microchip and said transmitter comprises a plurality of high density contact areas.

7. The device of claim 1, wherein said electronic connection between said microchip and said transmitter is wireless.

8. The device of claim 1, wherein said transmitter is affixed to a skull surface.

9. The device of claim 1, wherein said transmitter is a wireless transmitter.

10. The device of claim 1, wherein said device further comprises a base station, wherein said base station is electronically linked to said transmitter.

11. The device of claim 10, wherein said electronic connection between said base station and said transmitter comprises wires.

12. The device of claim 10, the electronic connection is wireless.

13. A method comprising;
a) providing;
i) a patient comprising a plurality of motor neurons; wherein said motor neurons exhibit neural data signals;
ii) a device comprising a biocompatible microchip comprising at least one microelectrode and a compressive spike sorting module, wherein said microchip is electronically linked to a transmitter;
b) implanting said microchip in said patient under conditions such that said neural data signals are recorded;
c) extracting a plurality of neural events from said recorded neural data signals;
d) formatting said plurality of neural events as a plurality of packets; and
e) transmitting said plurality of packets to said transmitter.

14. The method of claim 13, wherein said extracting is in real time.

15. The method of claim 13, wherein said formatting is in real time.

16. The method of claim 13, wherein said transmitting is in real time.

17. The method of claim 13, wherein said neural data comprises at least one neural spike.

18. The method of claim 17, wherein said at least one neural spike comprises at least one action potential.

19. The method of claim 13, wherein said packets comprise a channel index.

20. The method of claim 13, wherein said packets comprise a node index.

21. The method of claim 13, wherein said packets comprise a time index.

22. The method of claim 13, wherein said device further comprises a base station, wherein said base station is electronically linked to said transmitter.

23. The method of claim 22, wherein said method further comprises transmitting said plurality of packets to said base station.

24. The method of claim 13, wherein said neural data signals are compressed.

25. A method comprising;
a) providing;
i) a patient implanted with a biocompatible microchip, wherein said microchip comprises at least one microelectrode and a compressive spike sorting module, and wherein said microelectrode detects a plurality of neural signals;
ii) a transmitter electronically linked to said microchip; and
iii) a medical device in operable combination with the patient, wherein said medical device is electronically linked to said transmitter;
b) extracting a command signal from said plurality of neural signals; and
c) controlling said medical device in real time with said command signal.

26. The method of claim 25, wherein said controlling comprises moving said medical device.

27. The method of claim 25, wherein said controlling comprises activating said medical device.

28. The method of claim 25, wherein said command signal comprises a voluntary movement intention.

29. The method of claim 25, wherein said command signal comprises an involuntary movement intention.

30. The method of claim 25, wherein said electronic link between said microchip and said transmitter is wireless.

31. The method of claim 25, wherein said electronic link between said medical device and said transmitter is wireless.

32. The method of claim 25, wherein said microchip is implanted in the patient's brain.

33. The method of claim 32, wherein said patient's brain comprises an epileptic foci.

34. The method of claim 32, wherein said patient's brain comprises dopamine-depleted neurons.

35. The method of claim 25, wherein said medical device comprises an minipump.

36. The method of claim 35, wherein said minipump comprises a pharmaceutical compound.

37. The method of claim 25, wherein said medical device comprises a prosthetic.

38. The method of claim 37, wherein said prosthetic is an artificial arm.

39. The method of claim 37, wherein said prosthetic is an artificial leg.

40. The method of claim 37, wherein said prosthetic is an artificial hand.
